(19) United States
(12) Patent Application Publication
(10) Pub. No.: US 2017/0273961 A1
(43) Pub. Date: Sep. 28, 2017

(54) NOVEL IMINONITRILE DERIVATIVES

(71) Applicant: CURADEV PHARMA, PVT. LTD., Noida (IN)

(72) Inventors: Sandip Middya, Noida (IN); Dharmendra B Yadav, Noida (IN); Ritesh Shrivastava, Noida (IN); Sushil Raina, Noida (IN); Monali Banerjee, Noida (IN); Arjun Surya, Noida (IN)

(73) Assignee: CURADEV PHARMA, PVT. LTD., Noida (IN)

(21) Appl. No.: 15/503,964

(22) PCT Filed: Aug. 20, 2015
(86) PCT No.: PCT/IB2015/056311

§ 371 Date: Feb. 14, 2017

Related U.S. Application Data
(60) Provisional application No. 62/040,270, filed on Aug. 21, 2014.

(51) Int. Cl. A61K 31/4412; C07D 213/79; C07D 213/65; C07D 213/84; C07D 213/74

(52) U.S. Cl. CPC A61K 31/4412; C07D 213/84; C07D 213/74; C07D 213/65; C07D 213/79

(57) ABSTRACT

The invention relates to a compound of formula (I) wherein X¹ to X⁴ and R^{C} to R^{G} are defined as in the description and in the claims. The compound of formula (I) can be used as a medicament.

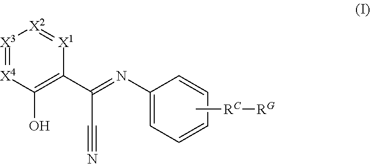

The present invention discloses novel iminonitrile compounds, pharmaceutical acceptable salts, isomers and pharmaceutical compositions thereof useful to treat conditions associated with indoleamine 2,3-dioxygenase. The invention also provides methods for preventing and/or treating medical conditions associated with indoleamine 2,3-dioxygenase in mammals, such as oncological disorders, neurodegenerative disorders, or autoimmune disorders, using the compounds and pharmaceutical compositions provided herein.

The invention relates in particular to a compound of formula (I)

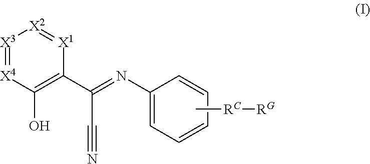

wherein
X¹ is CR¹, N, or NO;
X² is CR², N, or NO;
X³ is CR³, N, or NO;
X⁴ is CR⁴, N, or NO;
wherein at least one of X¹, X², X³ and X⁴ is N;
R¹, R², R³ and R⁴ are independently selected from the group consisting of H, optionally substituted C₁-C₆ alkyl, optionally substituted C₂-C₆ alkenyl, optionally substituted C₂-C₆ alkynyl, optionally substituted C₁-C₆ alkoxy, mono or bicyclic optionally substituted C₆-C₁₄ aryl, mono or bicyclic optionally substituted heteroaryl, optionally substituted (aryl)alkyl, (alkoxy)carbonyl, (alkyl)amido, (alkyl)amino, optionally substituted mono or bicyclic cycloalkyl, optionally substituted mono or bicyclic heterocyclyl, aminoalkyl, alkylcarboxyl, (alkyl)carboxyamido, optionally substituted (aryl)amino, hydroxyl, halogen, C₁-C₆haloalkyl, optionally substituted heterocyclyl(alkyl)-, optionally substituted heteroaryl(alkyl), hydroxyalkyl, perfluoroalkyl, optionally substituted aryloxy, optionally substituted heteroaryloxy, optionally substituted C₃-C₈ cycloalkoxy, N(R⁵)₂, CN, NO₂, CO₂H, CONR^{A}R^{B}, S(O)ₙR⁵, and optionally substituted heterocyclyloxy having 1 to 2 heteroatoms selected from the group consisting of O, S(O)ₙ, and NR⁶;
n is 0 to 2;
R^{A} and R^{B} are independently selected from the group consisting of H, optionally substituted C₁-C₆ alkyl, optionally substituted mono or bicyclic C₆-C₁₄ aryl, optionally substituted mono or bicyclic heteroaryl, optionally substituted (aryl)alkyl, optionally substituted mono or bicyclic C₃-C₈ cycloalkyl, optionally substituted mono or bicyclic heterocyclyl, C₁-C₆ haloalkyl, optionally substituted heterocyclyl(alkyl), optionally substituted heteroaryl(alkyl), hydroxyalkyl, and perfluoroalkyl;
R⁵ is independently selected from the group consisting H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or heterocyclyloxy having 1 to 2 heteroatoms selected from the group consisting of O, S(O)ₙ, and NR⁶;
R⁶ is independently selected from the group consisting of H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or optionally substituted heterocyclyloxy; and
R^{C} to R^{G} are independently selected from the group consisting of H, halogen, C₁-C₆ haloalkyl, C₁-C₆ alkoxy, heterocycle, optionally substituted C₁-C₆ alkyl, C₃-C₈ cycloalkyl, CN, —O(aryl), C₂-C₆ alkynyl, C(O)C₁-C₆ alkyl, —O—C₁-C₆ haloalkyl, and optionally substituted aryl;
or an isomer thereof, or a metabolite thereof, or a pharmaceutically acceptable salt or ester thereof.

The essential amino acid Tryptophan (Trp) is catabolized through the kynurenine (KYN) pathway. The initial rate-limiting step in the kynurenine pathway is performed by heme-containing oxidoreductase enzymes, including tryptophan 2,3-dioxygenase (TDO), indoleamine 2,3-dioxygenase-1 (IDO1), and indoleamine 2,3-dioxygenase-2 (IDO2). IDO1 and IDO2 share very limited homology with TDO at the amino acid level and, despite having different molecular structures; each enzyme has the same biochemical activity in that they each catalyze tryptophan to form N-formylkynurenine. IDO1, IDO2, and/or TDO activity alter local tryptophan concentrations, and the build-up of kynurenine pathway metabolites due to the activity of these enzymes can lead to numerous conditions associated with immune suppression.

IDO1 and TDO are implicated in the maintenance of immunosuppressive conditions associated with the persistence of tumor resistance, chronic infection, HIV infection, malaria, schizophrenia, depression as well as in the normal phenomenon of increased immunological tolerance to prevent fetal rejection in utero. Therapeutic agents that inhibit IDO1, IDO2, and TDO activity can be used to modulate regulatory T cells and activate cytotoxic T cells in immunosuppressive conditions associated with cancer and viral infection (e.g. HIV-AIDS, HCV). The local immunosuppressive properties of the kynurenine pathway and specifically IDO1 and TDO have been implicated in cancer. A large proportion of primary cancer cells have been shown to overexpress IDO1. In addition, TDO has recently been implicated in human brain tumors.

The earliest experiments had proposed an anti-microbial role for IDO1, and suggested that localized depletion of tryptophan by IDO1 led to microbial death (Yoshida et al., Proc. Natl. Acad. Sci. USA, 1978, 75(8):3998-4000). Subsequent research led to the discovery of a more complex role for IDO1 in immune suppression, best exemplified in the case of maternal tolerance towards the allogeneic fetus where IDO1 plays an immunosuppressive role in preventing fetal rejection from the uterus. Pregnant mice dosed with a specific IDO1 inhibitor rapidly reject allogeneic fetuses through induction of T cells (Munn et al., Science, 1998, 281(5380):1191-3). Studies since then have established IDO1 as a regulator of certain disorders of the immune system and have discovered that it plays a role in the ability of transplanted tissues to survive in new hosts (Radu et al., Plast. Reconstr. Surg., 2007 June, 119(7):2023-8). It is believed that increased IDO1 activity resulting in elevated kynurenine pathway metabolites causes peripheral and ultimately, systemic immune tolerance. In-vitro studies suggest that the proliferation and function of lymphocytes are exquisitely sensitive to kynurenines (Fallarino et al., Cell Death and Differentiation, 2002, 9(10):1069-1077). The expression of IDO1 by activated dendritic cells suppresses immune response by mechanisms that include inducing cell cycle arrest in T lymphocytes, down regulation of the T lymphocyte cell receptor (TCR) and activation of regulatory T cells (T-regs) (Terness et al., J. Exp. Med., 2002, 196(4):447-457; Fallarino et al., J. Immunol., 2006, 176(11):6752-6761).

IDO1 is induced chronically by HIV infection and in turn increases regulatory T cells leading to immunosuppression in patients (Sci. Transl. Med., 2010; 2). It has been recently shown that IDO1 inhibition can enhance the level of virus specific T cells and concomitantly reduce the number of virus infected macrophages in a mouse model of HIV (Potula et al., 2005, Blood, 106(7):2382-2390). IDO1 activity has also been implicated in other parasitic infections. Elevated activity of IDO1 in mouse malaria models has also been shown to be abolished by in vivo IDO1 inhibition (Tetsutani K., et al., Parasitology. 2007 7:923-30.

More recently, numerous reports published by a number of different groups have focused on the ability of tumors to create a tolerogenic environment suitable for survival, growth and metastasis by activating IDO1 (Prendergast, Nature, 2011, 478(7368):192-4). Studies of tumor resistance have shown that cells expressing IDO1 can increase the number of regulatory T cells and suppress cytotoxic T cell responses thus allowing immune escape and promoting tumor tolerance.

Kynurenine pathway and IDO1 are also believed to play a role in maternal tolerance and immunosuppressive process to prevent fetal rejection in utero (Munn et al., Science, 1998, 281(5380):1191-1193). Pregnant mice dosed with a specific IDO1 inhibitor rapidly reject allogeneic foetuses through suppression of T cells activity (Munn et al., Science, 1998, 281(5380):1191-1193). Studies since then have established IDO1 as a regulator of immune-mediated disorders and suggest that it plays a role in the ability of transplanted tissues to survive in new hosts (Radu et al., Plast. Reconstr. Surg., 2007 June, 119(7):2023-8).

The local immunosuppressive properties of the kynurenine pathway and specifically IDO1 and TDO have been implicated in cancer. A large proportion of primary cancer cells overexpress IDO1 and/or TDO (Pilotte et al., Proc. Natl. Acad. Sci. USA, 2012, Vol. 109(7):2497-2502). Several studies have focused on the ability of tumors to create a tolerogenic environment suitable for survival, growth and metastasis by activating IDO1 (Prendergast, Nature, 2011, 478:192-4). Increase in the number of T-regs and suppression of cytotoxic T cell responses associated with dysregulation of the Kynurenine pathway by overexpression of IDO1 and/or TDO appears to result in tumor resistance and promote tumor tolerance.

Data from both clinical and animal studies suggest that inhibiting IDO1 and/or TDO activity could be beneficial for cancer patients and may slow or prevent tumor metastases (Muller et al., Nature Medicine, 2005, 11(3):312-319; Brody et al., Cell Cycle, 2009, 8(12):1930-1934; Witkiewicz et al., Journal of the American College of Surgeons, 2008, 206:849-854; Pilotte et al., Proc. Natl. Acad. Sci. USA, 2012, Vol. 109(7):2497-2502). Genetic ablation of the IDO1 gene in mice (IDO1−/−) resulted in decreased incidence of DMBA-induced premalignant skin papillomas (Muller et al., PNAS, 2008, 105(44):17073-17078). Silencing of IDO1 expression by siRNA or a pharmacological IDO1 inhibitor 1-methyl tryptophan enhanced tumor-specific killing (Clin. Cancer Res., 2009, 15(2). In addition, inhibiting IDO1 in tumor-bearing hosts improved the outcome of conventional chemotherapy at reduced doses (Clin. Cancer Res., 2009, 15(2)). Clinically, the pronounced expression of IDO1 found in several human tumor types has been correlated with negative prognosis and poor survival rate (Zou, Nature Rev. Cancer, 2005, 5:263-274; Zamanakou et al., Immunol. Lett. 2007, 111(2):69-75). Serum from cancer patients has higher kynurenine/tryptophan ratio, a higher number of circulating T-regs, and increased effector T cell apoptosis when compared to serum from healthy volunteers (Suzuki et al., Lung Cancer, 2010, 67:361-365). Reversal of tumoral immune resistance by inhibition of tryptophan 2,3-dioxygenase has been studied by Pilotte et al. (Pilotte et al., Proc. Natl. Acad. Sci. USA, 2012, Vol. 109(7):2497-2502). Thus, decreasing the rate of kynurenine production by inhibiting IDO1 and/or TDO may be beneficial to cancer patients.

IDO1 and IDO2 are implicated in inflammatory diseases. IDO1 knock-out mice don't manifest spontaneous disorders of classical inflammation and existing known small molecule inhibitors of IDO do not elicit generalized inflammatory reactions (Prendergast et al. Curr Med Chem. 2011; 18(15):2257-62). Rather, IDO impairment alleviates disease severity in models of skin cancers promoted by chronic inflammation, inflammation-associated arthritis and allergic airway disease. Moreover, IDO2 is a critical mediator of autoantibody production and inflammatory pathogenesis in autoimmune arthritis. IDO2 knock-out mice have reduced joint inflammation compared to wild-type mice due to decreased pathogenic autoantibodies and Ab-secreting cells (Merlo et al. J. Immunol. (2014) vol. 192(5) 2082-2090). Thus, inhibitors of IDO1 and IDO2 are useful in the treatment of arthritis and other inflammatory diseases.

Kynurenine pathway dysregulation and IDO1 and TDO play an important role in the brain tumors and are implicated in inflammatory response in several neurodegenerative disorders including multiple sclerosis, Parkinson's disease, Alzheimer's disease, stroke, amyotrophic lateral sclerosis, dementia (Kim et al., J. Clin. Invest, 2012, 122(8):2940-2954; Gold et al., J. Neuroinflammation, 2011, 8:17; Parkinson's Disease, 2011, Volume 2011). Immunosuppression induced by IDO1 activity and the Kynurenine metabolites in the brain may be treated with inhibitors of IDO1 and/or TDO. For example, circulating T-reg levels were found to be decreased in patient with glioblastoma treated with anti-viral agent inhibitors of IDO1 (Söderlund, et al., J. Neuroinflammation, 2010, 7:44).

Several studies have found Kynurenine pathway metabolites to be neuroactive and neurotoxic. Neurotoxic kynurenine metabolites are known to increase in the spinal cord of rats with experimental allergic encephalomyelitis (Chiarugi et al., Neuroscience, 2001, 102(3):687-95). The neurotoxic effects of Kynurenine metabolites is exacerbated by increased plasma glucose levels. Additionally, changes in the relative or absolute concentrations of the kynurenines have been found in several neurodegenerative disorders, such as Alzheimer's disease, Huntington's disease and Parkinson's disease, stroke and epilepsy (Németh et al., Central Nervous System Agents in Medicinal Chemistry, 2007, 7:45-56; Wu et al. 2013; PLoS One; 8(4)).

Neuropsychiatric diseases and mood disorders such as depression and schizophrenia are also said to have IDO1 and Kynurenine dysregulation. Tryptophan depletion and deficiency of neurotransmitter 5-hydroxytryptamine (5-HT) leads to depression and anxiety. Increased IDO1 activity decreases the synthesis of 5-HT by reducing the amount of Tryptopan availability for 5-HT synthesis by increasing Tryp catabolism via the kynurenine pathway (Plangar et al. (2012) Neuropsychopharmacol Hung 2012; 14(4): 239-244). Increased IDO1 activity and levels of both kynurenine and kynurenic acid have been found in the brains of deceased schizophrenics (Linderholm et al., Schizophrenia Bulletin (2012) 38: 426-432)). Thus, inhibition of IDO1, IDO1, and TDO may also be an important treatment strategy for patients with neurological or neuropsychiatric disease or disorders such as depression and schizophrenia as well as insomnia.

Kynurenine pathway dysregulation and IDO1 and/or TDO activity also correlate with cardiovascular risk factors, and kynurenines and IDO1 are markers for Atherosclerosis and other cardiovascular heart diseases such as coronary artery disease (Platten et al., Science, 2005, 310(5749):850-5, Wirlietner et al. Eur J Clin Invest. 2003 July; 33(7):550-4) in addition to kidney disease. The kynurenines are associated with oxidative stress, inflammation and the prevalence of cardiovascular disease in patients with end-stage renal disease (Pawlak et al., Atherosclerosis, 2009, (204) 1:309-314). Studies show that kynurenine pathway metabolites are associated with endothelial dysfunction markers in the patients with chronic kidney disease (Pawlak et al., Advances in Medical Sciences, 2010, 55(2):196-203).

There is a need in the art for compounds that are inhibitors of the indoleamine 2,3-dioxygenase-1 and/or indoleamine 2,3-dioxygenase-2 and/or tryptophan 2,3-dioxygenase pathway, as well as for methods for treating diseases that can benefit from such inhibition.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, n-butyl and tert.-butyl, in particular methyl and tert.-butyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. A particular “alkoxy” is methoxy.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl and cyclohexyl, cycloheptyl and cyclooctyl. A particular examples of “cycloalkyl” is cyclohexyl.

The terms “halogen” or “halo”, alone or in combination, signifies fluorine, chlorine, bromine or iodine and particularly fluorine, chlorine or bromine, more particularly fluorine and chlorine. The term “halo”, in combination with another group, denotes the substitution of said group with at least one halogen, particularly substituted with one to five halogens, particularly one to three halogens, i.e. one, two or three halogens. A particular “haloalkyl” is trifluoromethyl.

The terms “hydroxyl” and “hydroxy”, alone or in combination, signify the —OH group.

The term “amino”, alone or in combination, signifies the primary amino group (—NH₂), the secondary amino group (—NH—) or the tertiary amino group (—N—).

The term “oxy”, alone or in combination, signifies a group of the formula —O—.

The “carbonyl”, alone or in combination, signifies a group of the formula —C(O)—

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

“Pharmaceutically acceptable esters” means that the compound of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compound of general formula (I), similar to the metabolically labile esters, which are capable of producing the parent compound of general formula (I) in vivo, are within the scope of this invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

In one aspect, the present invention provides a compound of formula (I), its isomers pharmaceutical acceptable salts thereof, or a metabolite thereof wherein X¹—X⁴, and R^{C}—R^{G} are defined herein.

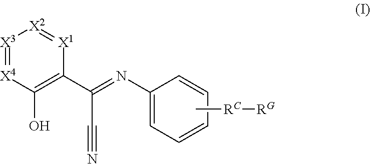

In another aspect, compounds of formulae (I-A), (I-B), (I-C), (I-D), and (I-E) are provided, wherein R^{C}-R^{G} and R¹-R⁴ are defined herein.

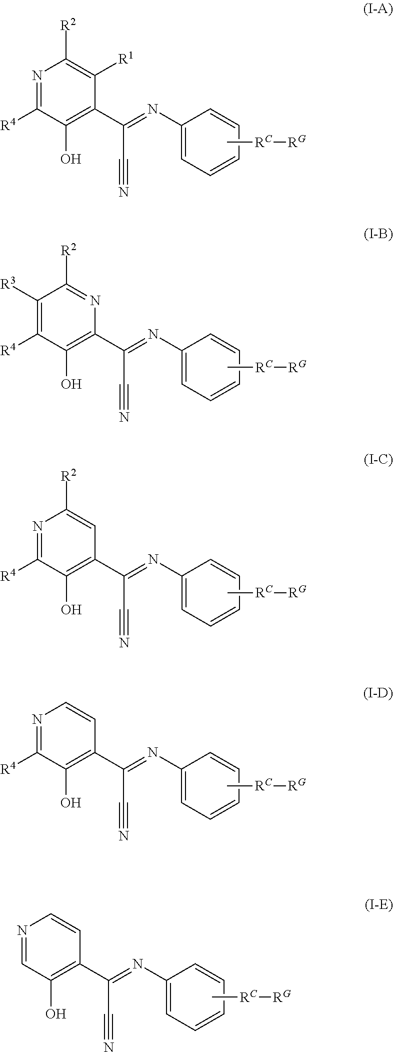

In one aspect, the invention relates to a metabolite of a compound of formula (I) or an isomer of said compound of formula (I), or a pharmaceutically acceptable salt thereof.

In a further aspect, a composition comprising a compound of formula (I) or an isomer or a pharmaceutically acceptable salt or a metabolite thereof as described herein and a pharmaceutically acceptable carrier is provided.

In another aspect, a method for treating a disease treatable by inhibiting a kynurenine pathway is provided and includes administering pharmaceutically effective amount of a compound of formula (I) or an isomer thereof, or a pharmaceutically acceptable salt or metabolite thereof to a subject in need thereof.

In another aspect, a method for regulating a kynurenine pathway is provided and includes administering pharmaceutically effective amount of a compound of formula (I) or an isomer thereof, or a pharmaceutically acceptable salt or metabolite thereof as described herein to a subject in need thereof.

In another aspect, a method of regulating one or more of indoleamine 2,3-dioxygenase-1 or an indoleamine 2,3-dioxygenase-2 or a tryptophan 2,3-dioxygenase enzymes is provided and includes administering pharmaceutically effective amount of a compound of formula (I) or an isomer thereof, or a pharmaceutically acceptable salt or metabolite thereof as described herein to a subject in need thereof.

In one aspect, a method of reducing kynurenine pathway metabolites is provided and includes administering pharmaceutically effective amount of a compound of formula (I) or an isomer thereof, or a pharmaceutically acceptable salt or metabolite thereof as described herein to a subject in need thereof.

In another aspect, a method of altering tryptophan levels in a subject and includes administering pharmaceutically effective amount of a compound of formula (I) or an isomer thereof, or a pharmaceutically acceptable salt or metabolite thereof described herein is provided. In one aspect, the tryptophan levels are increased. In another aspect, kynurenine/tryptophan ratio is decreased.

In one aspect, a method of treating a disease associated with or resulting from dysregulation of a kynurenine pathway is provided and includes administering pharmaceutically effective amount of a compound of formula (I) or an isomer thereof, or a pharmaceutically acceptable salt or metabolite thereof as described herein to a subject in need thereof.

In another aspect, a method for treating a disease caused by the dysregulation of the kynurenine pathway by inhibiting indoleamine 2,3-dioxygenase-1 and/or indoleamine 2,3-dioxygenase-2 and/or tryptophan 2,3-dioxygenase is provided and includes administering pharmaceutically effective amount of a compound of formula (I) or an isomer thereof, or a pharmaceutically acceptable salt or metabolite thereof described herein to a subject in need thereof.

In another aspect, a method for treating a disease associated with any one or more of indoleamine 2,3-dioxygenase-1 or indoleamine 2,3-dioxygenase-2 or tryptophan 2,3-dioxygenase enzymes is provided and includes administering pharmaceutically effective amount of a compound of formula (I) or a metabolite of the compound, or a pharmaceutically acceptable salt or isomers thereof described herein to a subject in need thereof.

In one aspect, the list of diseases comprises cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease, or cardiovascular disease. In another aspect, all foregoing methods comprise administration of one or more therapeutic agent or therapy. In one aspect, the therapeutic agent is a chemotherapeutic agent selected from a group further comprising a cancer vaccine, a targeted drug, a targeted antibody, an antibody fragment, an antimetabolite, an antineoplastic, an antifolate, a toxin, an alkylating agent, a DNA strand breaking agent, a DNA minor groove binding agent, a pyrimidine analogue, a purine analogue, a ribonucleotide reductase inhibitor, a tubulin interactive agent, an anti-hormonal agent, an immunomoldulator, an anti-adrenal agent, a cytokine, a radiation therapy, a cell therapy, or a hormone therapy.

In another aspect, a method of treating depression, Alzheimer's disease, dementia, schizophrenia, HIV infection, malaria, rheumatoid arthritis, insomnia or multiple sclerosis is provided and include administering a compound of formula (I) or an isomer thereof, or a pharmaceutically acceptable salt or metabolite thereof described herein to a patient.

In another aspect, a method of preparing a compound of formula (I) is provided as described herein.

In yet another aspect, a method for diagnosing and treating a disease associated with kynurenine pathway or one or more of indoleamine 2,3-dioxygenase-1 or an indoleamine 2,3-dioxygenase-2 or a tryptophan 2,3-dioxygenase enzymes in a subject is provided and includes: (i) assaying a blood and/or tissue sample from a subject; (ii) determining the subject's blood and/or tissue tryptophan or Kynurenine concentration or both in the sample; (iii) optionally determining the subject's Kynurenine/tryptophan ratio; and (iv) administering a compound of formula (I) or an isomer thereof, or a pharmaceutically acceptable salt or metabolite thereof described herein to a subject.

In still another aspect, a method of monitoring a disease associated with kynurenine pathway or one or more of indoleamine 2,3-dioxygenase-1 or an indoleamine 2,3-dioxygenase-2 or a tryptophan 2,3-dioxygenase enzymes in a subject is provided and includes (i) dosing a subject having a disease associated with kynurenine pathway with a compound, (ii) analyzing a blood or tissue samples or both at one or more time points or continuously during a treatment regimen, (iii) determining a tryptophan and a kynurenine concentration in the blood or the tissue sample or both, (iv) optionally determining the subject's kynurenine/tryptophan ratio, and (v) adjusting the treatment regimen or dosage of the compound of formula (I).

In a further aspect, a method for diagnosing and treating a disease associated with kynurenine pathway or one or more of indoleamine 2,3-dioxygenase-1 or an indoleamine 2,3-dioxygenase-2 or a tryptophan 2,3-dioxygenase enzymes in a patient is provided and includes (i) analyzing a patient sample for the presence or absence of altered Kynurenine/tryptophan ratio, wherein the patient is diagnosed with a disease associated with kynurenine pathway if altered kynurenine/tryptophan ratio is detected and (ii) administering a compound of formula (I) to the diagnosed patient.

In still a further aspect, a method for treating a disease associated with kynurenine pathway or one or more of an indoleamine 2,3-dioxygenase-1 or an indoleamine 2,3-dioxygenase-2 or a tryptophan 2,3-dioxygenase enzyme in a patient and includes (i) requesting a test providing the results of an analysis to determine whether the patient's kynurenine levels are altered, and (ii) administering a compound of formula (I) to the patient if the patient's kynurenine levels are altered.

Other aspects and advantages of the invention will be readily apparent from the following detailed description of the invention.

The invention provides compounds of formula (I) and isomers thereof, or pharmaceutically acceptable salts, and metabolites thereof, and pharmaceutical composition thereof, which are capable of reducing or eliminating immune-mediated disorders as standalone therapy (monotherapy) or in combination with other therapies, including without limitation, antiviral therapy, anti-inflammation therapy, conventional chemotherapy, or in combination with anti-cancer vaccines or in combination with hormonal therapy to slow or prevent various conditions or diseases including tumour growth. The invention further provides compounds and compositions which function by decreasing levels of kynurenine and/or altering the levels of tryptophan in plasma and/or tissues through the inhibition of the enzymes indoleamine 2,3-dioxygenase-1 (IDO1) or indoleamine 2,3-dioxygenase-2 (IDO2) or tryptophan 2,3-dioxygenase (TDO) or any combination of the three enzymes.

In one aspect, the present invention provides a compound of formula (I), its isomers pharmaceutical acceptable salts thereof, or a metabolite thereof,

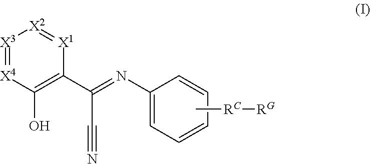

wherein
X¹ is CR¹, N, or NO; X² is CR², N, or NO; X³ is CR³, N, or NO; X⁴ is CR⁴, N, or NO and at least one of X¹, X², X³ and X⁴ is N; and
R¹, R², R³ and R⁴ are independently selected from the group consisting of H, optionally substituted C₁-C₆ alkyl, optionally substituted C₂-C₆ alkenyl, optionally substituted C₂-C₆ alkynyl, optionally substituted C₁-C₆ alkoxy, mono or bicyclic optionally substituted C₆-C₁₄ aryl, mono or bicyclic optionally substituted heteroaryl, optionally substituted (aryl)alkyl, (alkoxy)carbonyl, (alkyl)amido, (alkyl)amino, optionally substituted mono or bicyclic cycloalkyl, optionally substituted mono or bicyclic heterocyclyl, aminoalkyl, alkylcarboxyl, (alkyl)carboxyamido, optionally substituted (aryl)amino, hydroxyl, halogen, C₁-C₆haloalkyl, optionally substituted heterocyclyl(alkyl)-, optionally substituted heteroaryl(alkyl), hydroxyalkyl, perfluoroalkyl, optionally substituted aryloxy, optionally substituted heteroaryloxy, optionally substituted C₃-C₈ cycloalkoxy, N(R⁵)₂, CN, NO₂, CO₂H, CONR^{A}R^{B}, S(O)ₙR⁵, and optionally substituted heterocyclyloxy having 1 to 2 heteroatoms selected from the group consisting of O, S(O)ₙ, and NR⁶;
wherein R^{A}-R^{G}, R⁵ and n are as defined herein.

In one embodiment, at least one of X¹ is CR¹, X² is CR², X³ is CR³, and X⁴ is CR⁴.

In one embodiment, R¹ is H, halogen, CN, C₁-C₆ hydroxyalkyl, C₁-C₆ alkoxy, or C₁-C₆ alkyl. In another embodiment, R¹ is H. In yet another embodiment, the R¹ is a halogen. In still another embodiment, R¹ is a Cl. In yet another embodiment, R¹ is a methoxy or a methyl. In still another embodiment, R¹ is CN.

In a further embodiment, R² is H, halogen, hydroxyl, CN, N(R⁵)₂, mono or bicyclic optionally substituted C₆-C₁₄ aryl, optionally substituted C₁-C₆ alkoxy, optionally substituted C₁-C₆ alkyl, or optionally substituted aryloxy. In a still further embodiment, R² is F, Cl, Br, or I. In yet another embodiment, R² is H or optionally substituted C₁-C₆ alkyl. In yet another embodiment, R² is optionally substituted C₁-C₆ alkoxy or optionally substituted aryloxy. In still another embodiment, R² is N(R⁵)₂ or mono or bicyclic optionally substituted C₆-C₁₄ aryl. In another embodiment, R² is halogen.

In one embodiment, R³ is selected from group consisting of H, halogen, hydroxyl, NO₂ or CN, N(R⁵)₂, mono or bicyclic optionally substituted C₆-C₁₄ aryl, optionally substituted C₁-C₆ alkoxy, optionally substituted C₁-C₆ alkyl and optionally substituted aryloxy.

In another, R³ is selected from H, halogen and CN.

In another embodiment, R³ is H, halogen, NO₂ or CN. In still a further embodiment, R³ is H. In yet another embodiment, R³ is NO₂ or CN.

In yet another embodiment, R³ is N(R⁵)₂, mono or bicyclic optionally substituted C₆-C₁₄ aryl, optionally substituted C₁-C₆ alkoxy, optionally substituted C₁-C₆ alkyl, or optionally substituted aryloxy.

In yet another embodiment R⁴ is H, halogens, optionally substituted C₁-C₆ alkyl, optionally substituted C₂-C₆ alkenyl, optionally substituted C₂-C₆ alkynyl, optionally substituted C₁-C₆ alkoxy, mono or bicyclic optionally substituted C₆-C₁₄ aryl, CH₂-aryl, mono or bicyclic optionally substituted heteroaryl, optionally substituted (aryl)alkyl, 10 (alkoxy)carbonyl, (alkyl)amido, (alkyl)amino, optionally substituted mono or bicyclic cycloalkyl, optionally substituted mono or bicyclic heterocyclyl, aminoalkyl, alkylcarboxyl, (alkyl)carboxyamido, optionally substituted (aryl)amino, hydroxyl, halogen, C₁-C₆ haloalkyl, optionally substituted heterocyclyl(alkyl)-, optionally substituted heteroaryl(alkyl), hydroxyalkyl, perfluoroalkyl, optionally substituted aryloxy, optionally 15 substituted heteroaryloxy, optionally substituted C₃-C₈ cycloalkoxy, N(R⁵)₂, CN, NO₂, CO₂H, CONR^{A}R^{B}, S(O)ₙR⁵, and optionally substituted heterocyclyloxy having 1 to 2 heteroatoms selected from the group consisting of O, S(O)ₙ, and NR⁶, and n is 0 to 2.

In yet a further embodiment, R⁴ is H, halogen or CN. In still another embodiment, R⁴ is optionally substituted phenyl. In a further embodiment, R⁴ is phenyl substituted with one or more C₁-C₆ alkoxy or halogen. In a further embodiment, R⁴ is phenyl substituted with F, Cl, Br or I. In another embodiment, R⁴ is halogen.

In another embodiment, R⁴ is optionally substituted alkyl, optionally substituted cycloalkyl, or optionally substituted arylalkyl. In still another embodiment, R⁴ is N(R⁵)₂. In yet another embodiment, R⁴ is optionally substituted arylalkenyl or optionally substituted arylalkynyl. In still another embodiment, R⁴ is optionally substituted diarylamine or optionally substituted diphenylamine. In a further embodiment, R⁴ is optionally substituted aryl, optionally substituted bicylic aryl, heteroaryl, optionally substituted heteroaryl, or bicyclic heteroaryl. In a still further embodiment, R⁴ is an optionally substituted heterocyclyl.

In another embodiment, R⁴ is optionally substituted pyridine, optionally substituted picolyl, optionally substituted picolinamide. In yet another embodiment, R⁴ is optionally substituted (alkyl)carboxyamido, (aryl)carboxyamido, (alkyl)amido, alkylcarboxyl, (alkoxy)carbonyl, COOH, C₁-C₆ cyclyloxy, heterocyclyloxy, aryloxy, heteroaryloxy, perfluoroalkyl, S(O)ₙN(R⁵)₂, or pyrimidine. In a further embodiment, R⁴ is optionally substituted pyridine.

In a further embodiment, R⁵ is H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or heterocyclyloxy having 1 to 2 heteroatoms selected from the group consisting of O, S(O)ₙ, and NR⁶.

R⁶ is H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or optionally substituted heterocyclyloxy.

R^{A} and R^{B} are independently selected from the group consisting of H, optionally substituted C₁-C₆ alkyl, optionally substituted mono or bicyclic C₆-C₁₄ aryl, optionally substituted mono or bicyclic heteroaryl, optionally substituted (aryl)alkyl, optionally substituted mono or bicyclic C₃-C₈ cycloalkyl, optionally substituted mono or bicyclic heterocyclyl, C₁-C₆haloalkyl, optionally substituted heterocyclyl(alkyl), optionally substituted heteroaryl(alkyl), hydroxyalkyl and perfluoroalkyl.

n is 0 to 2. In one embodiment, n is 0. In another embodiment, n is 1. In a further embodiment, n is 2.

In one embodiment, R^{C} to R^{G} are defined with the following structure,

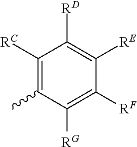

wherein, R^{C} to R^{G} are independently selected from among H, halogen, C₁-C₆ haloalkyl, C₁-C₆ alkoxy, heterocycle, optionally substituted C₁-C₆ alkyl, C₃-C₈ cycloalkyl, CN, —O(aryl), C₂-C₆ alkynyl, C(O)C₁-C₆ alkyl, —O—C₁-C₆ haloalkyl, and optionally substituted aryl.

In a further embodiment, R^{C} to R^{G} are independently selected from among H, halogen, CF₃, CHF₂, C(CH₃)F₂, OCF₃, OCH₃, OCH(CH₃)₂, morpholine, piperidine, CH₃, C(CH₃)₃, CH₂CH₃, CH(CH₃)₂, cyclopropyl, cyclohexyl, CH₂-cyclopropyl, CH₂-cyclobutyl, benzyl, CN, phenoxy, ethynyl, C(O)CH₃ and phenyl.

In yet another embodiment, R^{C} to R^{G} are independently selected from the group consisting of H and optionally substituted aryl. In one embodiment, R^{C} to R^{G} is independently selected from among H and aryl substituted with one or more halogen. In yet another embodiment, each halogen is independently selected from F, Cl, Br, or I. In another embodiment, R^{C} to R^{G} is independently selected from among H and aryl substituted with one or more Cl or F.

In one embodiment, R^{C} to R^{G} are independently selected from halogens.

In one embodiment, R^{C} to R^{G} are independently selected from Cl and F.

In one embodiment, the compound of formula (I) is selected from:
N-(3-Chloro-4-fluorophenyl)-3-hydroxyisonicotinimidoyl nitrile;
N-(3-Chloro-4-fluorophenyl)-2-cyano-3-hydroxyisonicotinimidoyl nitrile;
2-Cyano-N-(4-fluoro-3-(trifluoromethyl)phenyl)-3-hydroxyisonicotinimidoyl nitrile;
N-(3-Chloro-4-fluorophenyl)-3-hydroxy-[2,4′-bipyridine]-4-carbimidoyl nitrile;
N-(4-Fluoro-3-(trifluoromethyl)phenyl)-3-hydroxyisonicotinimidoyl nitrile;
N-(3-Chloro-4-fluorophenyl)-2-fluoro-5-hydroxyisonicotinimidoyl nitrile;
N-(3-Chloro-4-fluorophenyl)-3-hydroxy-2′-(methylcarbamoyl)-[2,4′-bipyridine]-4-carbimidoyl nitrile;
N-(3,4-Difluorophenyl)-3-hydroxy-[2,4′-bipyridine]-4-carbimidoyl nitrile;
N-(3-Chloro-4-fluorophenyl)-3-hydroxy-2′-methyl-[2,4′-bipyridine]-4-carbimidoyl nitrile;
N-(3,4-Difluorophenyl)-3-hydroxy-2′-methyl-[2,4′-bipyridine]-4-carbimidoyl nitrile;
N-(4-fluorophenyl)-3-hydroxy-2′-methyl-[2,4′-bipyridine]-4-carbimidoyl nitrile; and
N-(3-chloro-4-fluorophenyl)-3-hydroxy-2-(phenylamino) isonicotinimidoyl nitrile.

In another embodiment, the compound is of formula (I-A).

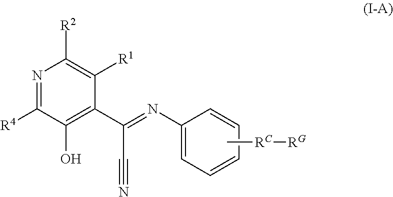

In a further embodiment, the compound is of formula (I-B).

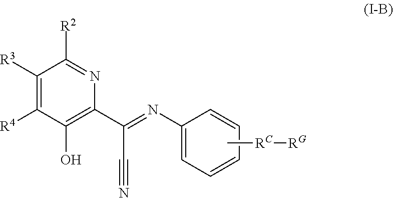

In yet another embodiment, the compound is of formula (I-C).

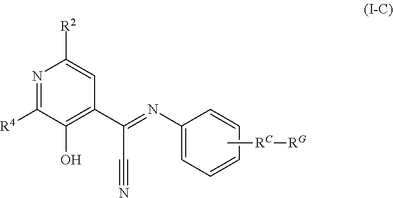

In still a further embodiment, the compound is of formula (I-D).

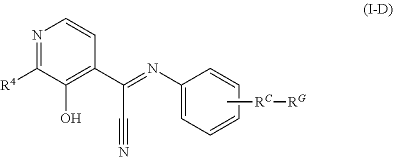

In another embodiment, the compound is of formula (I-E).

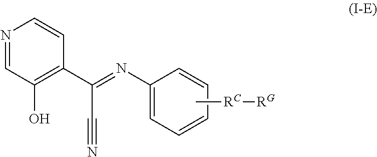

In (I-A) to (I-E), R¹-R⁴ and R^{C}-R^{G} are as defined herein.

The invention is in particular directed to:

A compound according of formula (I-F)

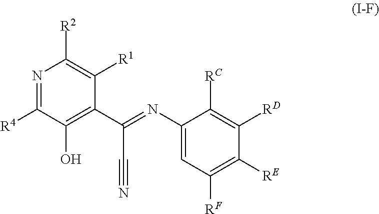

wherein
R¹ is hydrogen or halogen;
R² is hydrogen, halogen, alkyl or alkoxy;
R⁴ is hydrogen, halogen, alkyl, cycloalkyl, cyano, pyridinyl, alkylpyridinyl, alkylaminocarbonylpyridinyl, alkoxypyridinyl, alkylpyridinyl, halopyridinyl, morpholinylpyridinyl, haloalkylpyridinyl, phenyl, halohydroxyphenyl, halophenyl, phenylamino, diphenylamino, aminocarbonylphenyl, naphthyl, benzo[d][1,3]dioxolyl, morpholinyl, alkylpyrazolyl or alkylpyrimidinyl;
R^{C} is hydrogen or halogen;
R^{D} is hydrogen, halogen or haloalkyl;
R^{E} is hydrogen or halogen; and
R^{F} is hydrogen or halogen;
or a pharmaceutically acceptable salt or ester thereof;

A compound of formula (I-F) wherein R¹ is hydrogen or fluoro;

A compound of formula (I-F) wherein R¹ is hydrogen;

A compound of formula (I-F) wherein R² is hydrogen, fluoro, methyl or methoxy;

A compound of formula (I-F) wherein R² is hydrogen;

A compound of formula (I-F) wherein R⁴ is hydrogen, bromo, methyl, cyclohexyl, cyano, pyridinyl, methylpyridinyl, ethylpyridinyl, tert.-butylpyridinyl, methylaminocarbonylpyridinyl, n-butylaminocarbonylpyridinyl, tert.-butylaminocarbonylpyridinyl, methoxypyridinyl, dimethylpyridinyl, fluoropyridinyl, difluoropyridinyl, morpholinylpyridinyl, trifluoromethylpyridinyl, phenyl, fluorophenyl, fluorohydroxyphenyl, chlorofluorophenyl, phenylamino, diphenylamino, aminocarbonylphenyl, naphthyl, benzo[d][1,3]dioxolyl, morpholinyl, methylpyrazolyl or methylpyrimidinyl;

A compound of formula (I-F) wherein R⁴ is alkylpyridinyl;

A compound of formula (I-F) wherein R⁴ is methylpyridinyl;

A compound of formula (I-F) wherein R⁴ is alkylpyridinyl or alkylaminocarbonylpyridinyl;

A compound of formula (I-F) wherein R⁴ is methylpyridinyl or methylaminocarbonylpyridinyl;

A compound of formula (I-F) wherein R^{C} is hydrogen, chloro or fluoro;

A compound of formula (I-F) wherein R^{C} is hydrogen;

A compound of formula (I-F) wherein R^{D} is hydrogen, chloro, fluoro or trifluoromethyl;

A compound of formula (I-F) wherein R^{D} is hydrogen or halogen;

A compound of formula (I-F) wherein R^{D} is hydrogen, chloro or fluoro;

A compound of formula (I-F) wherein R^{E} is halogen;

A compound of formula (I-F) wherein R^{E} is hydrogen or fluoro;

A compound of formula (I-F) wherein R^{E} is fluoro;

A compound of formula (I-F) wherein R^{F} is hydrogen, chloro or fluoro;

A compound of formula (I-F) wherein R^{F} is hydrogen;

A compound of formula (I-F) wherein R^{D} and R^{E} are both halogen and R^{C} and R^{F} are both hydrogen; and

A compound selected from
N-(3-Chloro-4-fluorophenyl)-3-hydroxyisonicotinimidoyl nitrile;
N-(3-Chloro-4-fluorophenyl)-2-cyano-3-hydroxyisonicotinimidoyl nitrile;
2-Cyano-N-(4-fluoro-3-(trifluoromethyl)phenyl)-3-hydroxyisonicotinimidoyl nitrile;
N-(3-Chloro-4-fluorophenyl)-3-hydroxy-[2,4′-bipyridine]-4-carbimidoyl nitrile;
N-(4-Fluoro-3-(trifluoromethyl)phenyl)-3-hydroxyisonicotinimidoyl nitrile;
N-(3-Chloro-4-fluorophenyl)-2-fluoro-5-hydroxyIsonicotinimidoyl nitrile;
N-(3-Chloro-4-fluorophenyl)-3-hydroxy-2′-(methylcarbamoyl)-[2,4′-bipyridine]-4-carbimidoyl nitrile;
N-(3,4-Difluorophenyl)-3-hydroxy-[2,4′-bipyridine]-4-carbimidoyl nitrile;
N-(3-Chloro-4-fluorophenyl)-3-hydroxy-2′-methyl-[2,4′-bipyridine]-4-carbimidoyl nitrile;
N-(3,4-Difluorophenyl)-3-hydroxy-2′-methyl-[2,4′-bipyridine]-4-carbimidoyl nitrile;
N-(4-Fluorophenyl)-3-hydroxy-2′-methyl-[2,4′-bipyridine]-4-carbimidoyl nitrile;
N-(3-Chloro-4-fluorophenyl)-3-hydroxy-2-(phenylamino)isonicotinimidoyl nitrile;
N-(3-Chloro-4-fluorophenyl)-3-hydroxy-2-phenylisonicotinimidoyl nitrile,
N-(3-Chloro-4-fluorophenyl)-5-hydroxy-2-methoxyisonicotinimidoyl nitrile;
N-(3-Chloro-4-fluorophenyl)-3-hydroxy-2′-methoxy-[2,4′-bipyridine]-4-carbimidoyl nitrile;
(N-(3-Chloro-4-fluorophenyl)-3-hydroxy-2′,6′-dimethyl-[2,4′-bipyridine]-4-carbimidoyl nitrile;
2-(Benzo[d][1,3]dioxol-5-yl)-N-(3-chloro-4-fluorophenyl)-3-hydroxyisonicotinimidoyl nitrile;
N-(3-Chloro-4-fluorophenyl)-3-hydroxy-2-methylisonicotinimidoyl nitrile;
2-Bromo-N-(3-chloro-4-fluorophenyl)-3-hydroxyIsonicotinimidoyl nitrile;
(N-(2-Chlorophenyl)-3-hydroxy-[2,4′-bipyridine]-4-carbimidoyl nitrile;
N-(3-Chloro-4-fluorophenyl)-2′,6′-difluoro-3-hydroxy-[2,4′-bipyridine]-4-carbimidoyl nitrile;
N-(3-Chlorophenyl)-3-hydroxy-[2,4′-bipyridine]-4-carbimidoyl nitrile;
N-(3-Chlorophenyl)-3-hydroxy-2′-methyl-[2,4′-bipyridine]-4-carbimidoyl nitrile;
3-Hydroxy-2′-methyl-N-phenyl-[2,4′-bipyridine]-4-carbimidoyl nitrile;
N-(2-Chlorophenyl)-3-hydroxy-2′-methyl-[2,4′-bipyridine]-4-carbimidoyl nitrile;
N-(3-Chloro-4-fluorophenyl)-3-hydroxy-2′,6-dimethyl-[2,4′-bipyridine]-4-carbimidoyl nitrile;
N-(2,4-Difluorophenyl)-3-hydroxy-2′-methyl-[2,4′-bipyridine]-4-carbimidoyl nitrile;
3-Hydroxy-N-(3-(trifluoromethyl)phenyl)-[2,4′-bipyridine]-4-carbimidoyl nitrile;
N-(3-Fluorophenyl)-3-hydroxy-2′-methyl-[2,4′-bipyridine]-4-carbimidoyl nitrile;
N-(3-Chlorophenyl)-3-hydroxy-2′-(methylcarbamoyl)-[2,4′-bipyridine]-4-carbimidoyl nitrile;
N-(4-Fluoro-3-(trifluoromethyl)phenyl)-3-hydroxy-2′-(methylcarbamoyl)-[2,4′-bipyridine]-4-carbimidoyl nitrile;
N-(4-Fluorophenyl)-3-hydroxy-2′-(methylcarbamoyl)-[2,4′-bipyridine]-4-carbimidoyl nitrile;
N-(3-Chlorophenyl)-3-hydroxyisonicotinimidoyl nitrile;
3-Hydroxy-N-(3-(trifluoromethyl)phenyl)isonicotinimidoyl nitrile;
N-(3-Fluorophenyl)-3-hydroxyisonIcotinimidoyl nitrile;
N-(3,4-Difluorophenyl)-3-hydroxyisonicotinimidoyl nitrile;
N-(3,4-Difluorophenyl)-3-hydroxy-2′-(methylcarbamoyl)-[2,4′-bipyridine]-4-carbimidoyl nitrile;
3-Hydroxy-2′-methyl-N-(3-(trifluoromethyl)phenyl)-[2,4′-bipyridine]-4-carbimidoyl nitrile;
N-(4-Fluoro-3-(trifluoromethyl)phenyl)-3-hydroxy-2′-methyl-[2,4′-bipyridine]-4-carbimidoyl nitrile;
N-(4-Fluoro-3-(trifluoromethyl)phenyl)-3-hydroxy-[2,4′-bipyridine]-4-carbimidoyl nitrile;
N-(3-Fluorophenyl)-3-hydroxy-[2,4′-bipyridine]-4-carbimidoyl nitrile;
3-Hydroxy-2-phenyl-N-(3-(trifluoromethyl)phenyl)isonicotinimidoyl nitrile;
2′-Fluoro-N-(4-fluorophenyl)-3-hydroxy-[2,4′-bipyridine]-4-carbimidoyl nitrile
N-(3-Chloro-4-fluorophenyl)-3-hydroxy-[2,3′-bipyridine]-4-carbimidoyl nitrile;
N-(3,4-Difluorophenyl)-3-hydroxy-2-phenylisonicotinimidoyl nitrile;
N-(3-Chloro-4-fluorophenyl)-3-hydroxy-2-(naphthalen-1-yl)isonicotinimidoyl nitrile;
N-(3-Chloro-4-fluorophenyl)-2-(diphenylamino)-3-hydroxyisonicotinimidoyl nitrile;
N-(3-Chloro-4-fluorophenyl)-6-fluoro-3-hydroxy-2′-methyl-[2,4′-bipyridine]-4-carbimidoyl nitrile;
2′-(tert-Butyl)-N-(3-chloro-4-fluorophenyl)-3-hydroxy-[2,4′-bipyridine]-4-carbimidoyl nitrile;
N-(2-Chlorophenyl)-3-hydroxy-[2,4′-bipyridine]-4-carbimidoyl nitrile;
N-(3-Chloro-4-fluorophenyl)-3-hydroxy-2-morpholinoisonicotinimidoyl nitrile;
N-(3-Chloro-4-fluorophenyl)-2-(4-fluoro-3-hydroxyphenyl)-3-hydroxyisonicotinimidoyl nitrile;
N-(3,4-Difluorophenyl)-6-fluoro-3-hydroxy-2′-methyl-[2,4′-bipyridine]-4-carbimidoyl nitrile;
N-(3-Chloro-4-fluorophenyl)-3-hydroxy-2′-morpholino-[2,4′-bipyridine]-4-carbimidoyl nitrile;
6-Fluoro-N-(4-fluorophenyl)-3-hydroxy-2′-methyl-[2,4′-bipyridine]-4-carbimidoyl nitrile;
N-(3-Chloro-4-fluorophenyl)-2-(2-chloro-5-fluorophenyl)-3-hydroxyisonicotinimidoyl nitrile;
N-(3-Chloro-4-fluorophenyl)-3-hydroxy-2-(naphthalen-2-yl)isonicotinimidoyl nitrile;
N-(3-Chloro-4-fluorophenyl)-3-hydroxy-2′-(trifluoromethyl)-[2,4′-bipyridine]-4-carbimidoyl nitrile;
N-(3-Chloro-4-fluorophenyl)-2′-ethyl-3-hydroxy-[2,4′-bipyridine]-4-carbimidoyl nitrile;
2′-(tert-Butylcarbamoyl)-N-(3-chloro-4-fluorophenyl)-3-hydroxy-[2,4′-bipyridine]-4-carbimidoyl nitrile;
2′-(Butylcarbamoyl)-N-(3-chloro-4-fluorophenyl)-3-hydroxy-[2,4′-bipyridine]-4-carbimidoyl nitrile;
2-(4-Carbamoylphenyl)-N-(3-chloro-4-fluorophenyl)-3-hydroxy-6-methoxyisonicotinimidoyl nitrile;
N-(3-Chloro-4-fluorophenyl)-2-(4-fluorophenyl)-3-hydroxy-6-methoxyisonicotinimidoyl nitrile;
N-(3-Chloro-4-fluorophenyl)-3-hydroxy-2-(1-methyl-1H-pyrazol-4-yl)isonicotinimidoyl c nitrile;
N-(3-Chloro-4-fluorophenyl)-2-cyclohexyl-3-hydroxyisonicotinimidoyl nitrile;
N-(3-Chloro-4-fluorophenyl)-3-hydroxy-6′-methyl-[2,3′-bipyridine]-4-carbimidoyl nitrile;
N-(3-Chloro-4-fluorophenyl)-3-hydroxy-2-(2-methylpyrimidin-5-yl)isonicotinimidoyl nitrile;
N-(3-Chloro-4-fluorophenyl)-3-hydroxy-2-(3-(methylcarbamoyl)phenyl)isonicotinimidoyl nitrile; and
N-(3-chloro-4-fluorophenyl)-3-fluoro-5-hydroxyisonicotinimidoyl nitrile.
The invention relates in particular to a compound selected from
N-(3-Chloro-4-fluorophenyl)-3-hydroxyisonicotinimidoyl nitrile;
N-(3-Chloro-4-fluorophenyl)-2-cyano-3-hydroxyisonicotinimidoyl nitrile;
2-Cyano-N-(4-fluoro-3-(trifluoromethyl)phenyl)-3-hydroxyisonicotinimidoyl nitrile;
N-(3-Chloro-4-fluorophenyl)-3-hydroxy-[2,4′-bipyridine]-4-carbimidoyl nitrile;
N-(4-Fluoro-3-(trifluoromethyl)phenyl)-3-hydroxyisonicotinimidoyl nitrile;
N-(3-Chloro-4-fluorophenyl)-2-fluoro-5-hydroxyIsonicotinimidoyl nitrile;
N-(3-Chloro-4-fluorophenyl)-3-hydroxy-2′-(methylcarbamoyl)-[2,4′-bipyridine]-4-carbimidoyl nitrile;
N-(3,4-Difluorophenyl)-3-hydroxy-[2,4′-bipyridine]-4-carbimidoyl nitrile;
N-(3-Chloro-4-fluorophenyl)-3-hydroxy-2′-methyl-[2,4′-bipyridine]-4-carbimidoyl nitrile;
N-(3,4-Difluorophenyl)-3-hydroxy-2′-methyl-[2,4′-bipyridine]-4-carbimidoyl nitrile;
N-(4-Fluorophenyl)-3-hydroxy-2′-methyl-[2,4′-bipyridine]-4-carbimidoyl nitrile;
N-(3-Chloro-4-fluorophenyl)-3-hydroxy-2-(phenylamino)isonicotinimidoyl nitrile;
N-(3-Chloro-4-fluorophenyl)-3-hydroxy-2-phenylisonicotinimidoyl nitrile;
N-(3-Chloro-4-fluorophenyl)-5-hydroxy-2-methoxyisonicotinimidoyl nitrile;
N-(3-Chloro-4-fluorophenyl)-3-hydroxy-2′-methoxy-[2,4′-bipyridine]-4-carbimidoyl nitrile;
(N-(3-Chloro-4-fluorophenyl)-3-hydroxy-2′,6′-dimethyl-[2,4′-bipyridine]-4-carbimidoyl nitrile;
2-(Benzo[d][1,3]dioxol-5-yl)-N-(3-chloro-4-fluorophenyl)-3-hydroxyisonicotinimidoyl nitrile;
N-(3-Chloro-4-fluorophenyl)-3-hydroxy-2-methylisonicotinimidoyl nitrile;
2-Bromo-N-(3-chloro-4-fluorophenyl)-3-hydroxyIsonicotinimidoyl nitrile;
(N-(2-Chlorophenyl)-3-hydroxy-[2,4′-bipyridine]-4-carbimidoyl nitrile;
N-(3-Chloro-4-fluorophenyl)-2′,6′-difluoro-3-hydroxy-[2,4′-bipyridine]-4-carbimidoyl nitrile;
N-(3-Chlorophenyl)-3-hydroxy-[2,4′-bipyridine]-4-carbimidoyl nitrile;
N-(3-Chlorophenyl)-3-hydroxy-2′-methyl-[2,4′-bipyridine]-4-carbimidoyl nitrile;
3-Hydroxy-2′-methyl-N-phenyl-[2,4′-bipyridine]-4-carbimidoyl nitrile;
N-(2-Chlorophenyl)-3-hydroxy-2′-methyl-[2,4′-bipyridine]-4-carbimidoyl nitrile;
N-(3-Chloro-4-fluorophenyl)-3-hydroxy-2′,6-dimethyl-[2,4′-bipyridine]-4-carbimidoyl nitrile;
N-(2,4-Difluorophenyl)-3-hydroxy-2′-methyl-[2,4′-bipyridine]-4-carbimidoyl nitrile;
3-Hydroxy-N-(3-(trifluoromethyl)phenyl)-[2,4′-bipyridine]-4-carbimidoyl nitrile;
N-(3-Fluorophenyl)-3-hydroxy-2′-methyl-[2,4′-bipyridine]-4-carbimidoyl nitrile;
N-(3-Chlorophenyl)-3-hydroxy-2′-(methylcarbamoyl)-[2,4′-bipyridine]-4-carbimidoyl nitrile;
N-(4-Fluoro-3-(trifluoromethyl)phenyl)-3-hydroxy-2′-(methylcarbamoyl)-[2,4′-bipyridine]-4-carbimidoyl nitrile;
N-(4-Fluorophenyl)-3-hydroxy-2′-(methylcarbamoyl)-[2,4′-bipyridine]-4-carbimidoyl nitrile;
N-(3-Chlorophenyl)-3-hydroxyisonicotinimidoyl nitrile;
3-Hydroxy-N-(3-(trifluoromethyl)phenyl)isonicotinimidoyl nitrile;
N-(3-Fluorophenyl)-3-hydroxyisonIcotinimidoyl nitrile,
N-(3,4-Difluorophenyl)-3-hydroxyisonicotinimidoyl nitrile;
N-(3,4-Difluorophenyl)-3-hydroxy-2′-(methylcarbamoyl)-[2,4′-bipyridine]-4-carbimidoyl nitrile;
3-Hydroxy-2′-methyl-N-(3-(trifluoromethyl)phenyl)-[2,4′-bipyridine]-4-carbimidoyl nitrile;
N-(4-Fluoro-3-(trifluoromethyl)phenyl)-3-hydroxy-2′-methyl-[2,4′-bipyridine]-4-carbimidoyl nitrile;
N-(4-Fluoro-3-(trifluoromethyl)phenyl)-3-hydroxy-[2,4′-bipyridine]-4-carbimidoyl nitrile;
N-(3-Fluorophenyl)-3-hydroxy-[2,4′-bipyridine]-4-carbimidoyl nitrile;
3-Hydroxy-2-phenyl-N-(3-(trifluoromethyl)phenyl)isonicotinimidoylnitrile;
2′-Fluoro-N-(4-fluorophenyl)-3-hydroxy-[2,4′-bipyridine]-4-carbimidoyl nitrile; and
N-(3-Chloro-4-fluorophenyl)-3-hydroxy-[2,3′-bipyridine]-4-carbimidoyl nitrile.

The compound of formula (I-F) is a particular sub-type of compound of formula (I).

The invention further relates to:

A compound of formula (I) wherein X¹ is CR¹, X² is CR², X³ is N and X⁴ is CR⁴;

A compound of formula (I) wherein R¹ is hydrogen or fluoro;

A compound of formula (I) wherein R¹ is hydrogen;

A compound of formula (I) wherein R² is hydrogen, fluoro, methyl or methoxy;

A compound of formula (I) wherein R² is hydrogen;

A compound of formula (I) wherein R⁴ is hydrogen, bromo, methyl, cyclohexyl, cyano, pyridinyl, methylpyridinyl, ethylpyridinyl, tert.-butylpyridinyl, methylaminocarbonylpyridinyl, n-butylaminocarbonylpyridinyl, tert.-butylaminocarbonylpyridinyl, methoxypyridinyl, dimethylpyridinyl, fluoropyridinyl, difluoropyridinyl, morpholinylpyridinyl, trifluoromethylpyridinyl, phenyl, fluorophenyl, fluorohydroxyphenyl, chlorofluorophenyl, phenylamino, diphenylamino, aminocarbonylphenyl, naphthyl, benzo[d][1,3]dioxolyl, morpholinyl, methylpyrazolyl or methylpyrimidinyl;

A compound of formula (I) wherein R⁴ is alkylpyridinyl;

A compound of formula (I) wherein R⁴ is methylpyridinyl;

A compound of formula (I) wherein R⁴ is alkylpyridinyl or alkylaminocarbonylpyridinyl;

A compound of formula (I) wherein R⁴ is methylpyridinyl or methylaminocarbonylpyridinyl;

A compound of formula (I) wherein R^{C} is hydrogen, chloro or fluoro;

A compound of formula (I) wherein R^{C} is hydrogen;

A compound of formula (I) wherein R^{D} is hydrogen, chloro, fluoro or trifluoromethyl;

A compound of formula (I) wherein R^{D} is hydrogen or halogen;

A compound of formula (I) wherein R^{D} is hydrogen, chloro or fluoro;

A compound of formula (I) wherein R^{E} is halogen;

A compound of formula (I) wherein R^{E} is hydrogen or fluoro;

A compound of formula (I) wherein R^{E} is fluoro;

A compound of formula (I) wherein R^{F} is hydrogen, chloro or fluoro;

A compound of formula (I) wherein R^{F} is hydrogen; and

A compound of formula (I) wherein R^{D} and R^{E} are both halogen and R^{C} and R^{F} are both hydrogen.

Also falling within the scope of this invention are in vivo metabolic products of the compounds of formula (I) described herein. Such metabolic products may result from the oxidation, reduction, hydrolysis, amidation, deamidation, esterification, deesterification, enzymatic cleavage, and the like, of the administered compound. Accordingly, compounds of the invention include without limitation metabolites of compounds of formula (I). Further, the invention includes metabolites of compounds of formula (I), including compounds produced synthetically and/or by a process comprising contacting a compound of this invention with a mammal or a cell, for example, a mammalian cell (including without limitation, rat, mice, human, ape, monkey, rabbit, guinea pig, hamster, pig, cow, goat, sheep, cat, dog etc.) or a eukaryotic cell such as a yeast cell, for a period of time sufficient to yield a metabolic product thereof.

Compounds of formula (I) or metabolites thereof that are basic in nature are capable of forming a wide variety of different salts with various inorganic and organic acids. Acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nirate, sulfate or bisulfate, phosphate or acid phosphate, acetate, lactate, citrate or acid citrate, tartarate or bitartrate, succinate, maleate, fumarate, gluconate, saccharate, benzoate, methanesulfonate and palmoate salts and the like. Compounds of the invention may also exist as hydrates or solvates.

Compounds of formula (I) or metabolites thereof which are also acidic in nature, e.g., where R¹-R⁴ and R^{C} to R^{G} includes a COOH or tetrazole moiety, are capable of forming base salts with various pharmacologically acceptable cation. Examples of such salts include the alkali metal or alkaline-earth metal salts and particularly, the sodium and potassium salts.

Compounds of formula (I) and isomers thereof, and pharmaceutically acceptable salts and metabolites thereof, are well within the scope of this invention.

The compounds of the invention may be synthesized by synthetic routes that include processes analogous to those well-known in the chemical arts and those included in the present application. Starting materials are generally available from commercial sources such as Sigma Aldrich Chemicals (Milwakee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Vogel's Textbook of Practical Organic Chemistry (5th Edition) A. I. Vogel et al., or Beilsteins Handbuch der organischen Chemi, 4, Aufl. Ed. Springer-Verlag, Berlin, including supplements (also available via the Beilstein and Reaxys online database).

Protection of functional groups (e.g., primary or secondary amines) of the intermediates may be necessary in preparing compounds of formula (I). The need for such protection will vary depending on the nature of the remote functionality and the conditions of the preparation methods. Suitable amino-protecting groups include acetyl, trifluoroacetyl, t-butoxycarbonyl (Boc), benzyloxycarbonyl (CBz) and 9-fluorenylethyleneoxycarbonyl (Fmoc). The hydroxyl-protecting groups include methoxymethyl chloride (MOMCl) or 2-(trimethylsilyl)ethoxymethyl chloride (SEMCl). The need for such protection is readily determined by one skilled in the art. For a general description of protecting groups and their use, see T. W. Greene, Protective groups in Organic Synthesis, John Wiley & Sons, New York, 1991.

Methods useful for making the compounds of formula (I) are set forth in the Examples below and generalized in Schemes 1-2. One of skill in the art will recognize that Schemes 1-2 can be adapted to produce other compounds of formula (I), isomers, metabolites, and pharmaceutically acceptable salts of compounds of formula (I) according to the present invention.

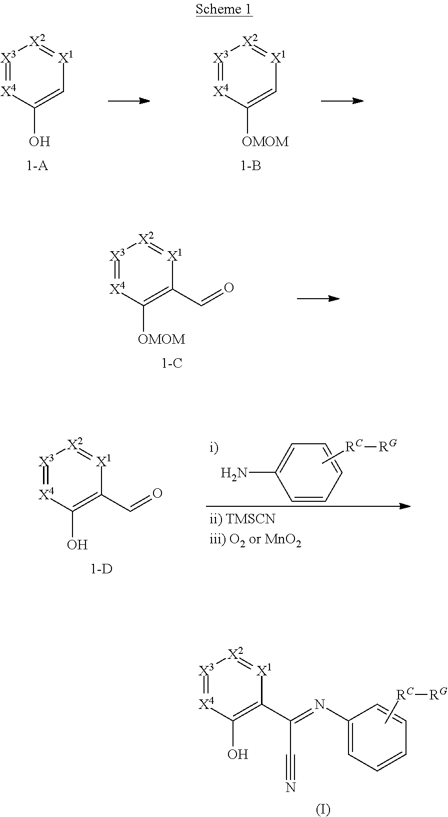

Scheme 1 provides the compounds of formula (I). A sodium or potassium alkoxide or NaH or Cs₂CO₃ was added to a solution of compound 1-A. In one embodiment, the potassium alkoxide was potassium tert-butoxide. Compound 1-A was reacted with methoxymethyl chloride or 2-(trimethylsilyl)ethoxymethyl chloride (SEMCl) to provide MOM or SEM protected compound 1-B. TMEDA, HMPA, TEA, or DIPEA was then added to a solution of compound 1-B, followed by addition of an alkyllithium reagent and then DMF, N-formylpiperidine or ethylformate to provide carbaldehyde 1-C. In one embodiment, the alkyl-lithium reagent was n-BuLi. Deprotection of the MOM or SEM group provided the 3-hydroxy carbaldehyde compound 1-D. In one embodiment the acid was TFA or HCl Compound 1-D was then treated in sequential manner with substituted aniline in the presence of an acid to provide imine intermediate which in-situ underwent Strecker reaction using nitrile ion source followed by oxidation to afford iminonitrile compound (I). In one embodiment the cyanide ion source was TMSCN or NaCN or KCN. In another embodiment the oxidizing agent was air. In yet another embodiment the oxidizing agent was MnO₂.

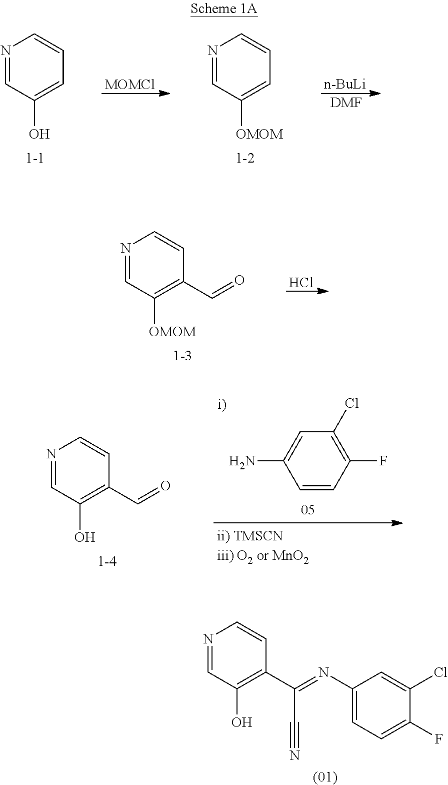

Scheme 1A provides the formation of compound N-(3-chloro-4-fluorophenyl)-3-hydroxyisonicotinimidoyl nitrile (01). Potassium tert-butoxide was added to 3-hydroxypyridine 1-1 in THF at low temperature and then methoxymethyl chloride was added to afford the desired MOM protected compound 3-(methoxymethoxy)pyridine 1-2. TMEDA was then added to a solution of compound 1-2 followed by addition of n-BuLi at −10 to −75° C. After 30 min. DMF was added to give the MOM protected carbaldehyde 3-(methoxymethoxy)isonicotinaldehyde 1-3. Deprotection of the MOM group provided 3-hydroxypyridine-2-carbaldehyde 1-4. In one embodiment, the deprotection was performed using 3N HCl. Compound 1-4 was treated in sequential manner with 3-chloro-4-fluoroaniline to provide imine intermediate which in-situ underwent Strecker reaction using TMSCN followed by oxidation with oxidizing agent MnO₂ or in the presence of oxygen to afford N-(3-chloro-4-fluorophenyl)-3-hydroxyisonicotinimidoyl nitrile (01).

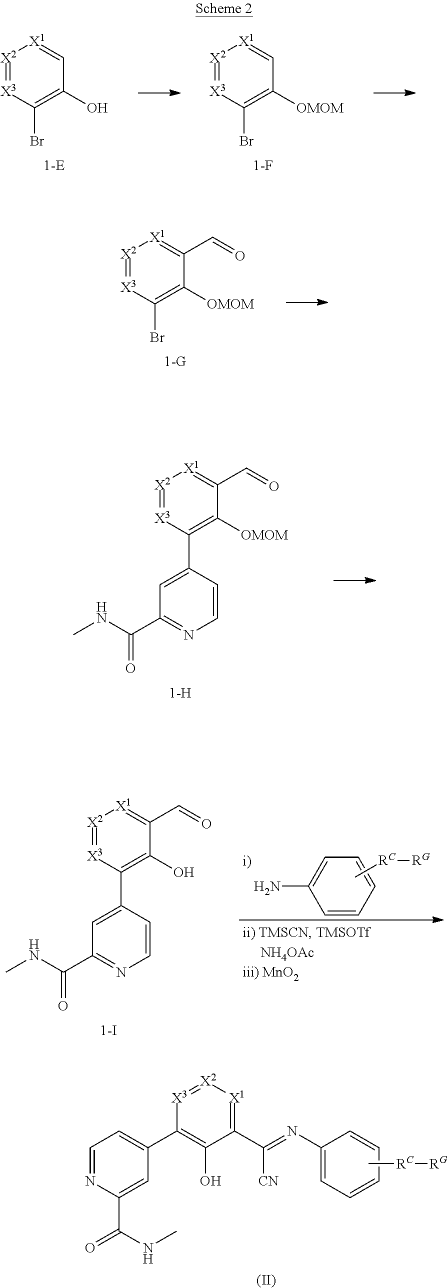

Scheme 2 describes the synthesis of compound (II). The starting bromohydroxy compound 1-E was subjected to MOM protection or SEM protection using MOMCl or SEMCl respectively in the presence of a base to provide the product 1-F which in turn was formylated with DMF or N-formylpiperidine in the presence of base like n-BuLi, s-BuLi, LDA, or LTMP at −78° C. to give product 1-G. The compound 1-G was coupled with a suitable substituted aryl- or heteroaryl boronic acid or ester under Suzuki cross-coupling reaction conditions to provide compound 1-H. In one embodiment, the boronic ester used was N-methyl-4-(4,4,5,5-tetramethyl-1,3,2-dioxaborolan-2-yl)picolinamide. In another embodiment, the coupling reaction was done in presence of tripotassium phosphate, tricyclohexylphosphine and Pd₂(dba)₃ in dioxane. The compound 1-H was deprotected in presence of a Lewis acid to provide 1-I. In one embodiment, the acid was TFA or HCl. Compound 1-D was then treated in sequential manner with substituted aniline in the presence of an Lewis acid to provide imine intermediate which in-situ underwent Strecker reaction using nitrile ion source followed by oxidation to afford iminonitrile compound (II). In one embodiment the cyanide ion source was TMSCN or NaCN or KCN. In another embodiment the oxidizing agent was air. In yet another embodiment the oxidizing agent was MnO₂. In another embodiment, the Lewis acid was TMSOTf.

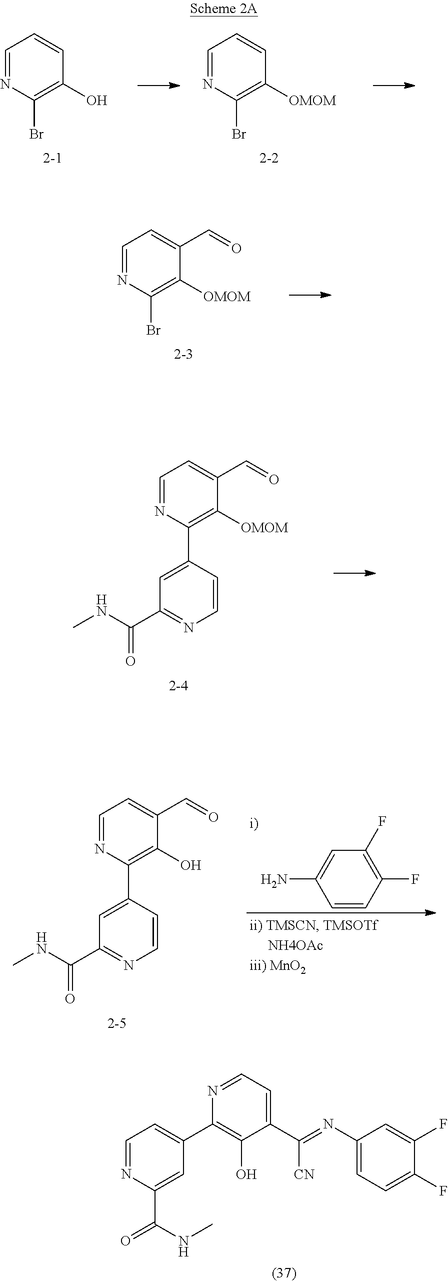

Scheme 2A depicts the synthesis of N-(3,4-difluorophenyl)-3-hydroxy-2′-(methylcarbamoyl)-[2,4′-bipyridine]-4-carbimidoyl nitrile 37. 2-Bromo-3-hydroxypyridine 2-1 was treated with MOMCl in the presence of t-BuOK in THF resulting in to the formation of 2-bromo-3-(methoxymethoxy)pyridine 2-2. The MOM protected compound underwent formylation with ethylformate or DMF in the presence of LDA or n-BuLi in THF at −78° C. to give 2-bromo-3-(methoxymethoxy)isonicotinaldehyde 2-3. The compound 2-3 was coupled with N-methyl-4-(4,4,5,5-tetramethyl-1,3,2-dioxaborolan-2-yl)picolinamide under Suzuki cross coupling condition using tripotassium phosphate, tricyclohexylphosphine and Pd₂(dba)₃ in dioxane to afford 4-formyl-3-(methoxymethoxy)-N-methyl-[2,4′-bipyridine]-2′-carboxamide 2-4 which in turn underwent MOM-de-protection with TFA-DCM to form 4-formyl-3-hydroxy-N-methyl-[2,4′-bipyridine]-2′-carboxamide 2-5. The compound 2-5 was coupled with 3,4-difluoroaniline to form intermediate imine which was treated in-situ with TMSCN followed by TMSOTf and NH₄OAc buffer solution at 40 C for overnight. The isolated compound was further oxidation with oxidizing agent MnO₂ or in the presence of oxygen to afford N-(3,4-difluorophenyl)-3-hydroxy-2′-(methylcarbamoyl)-[2,4′-bipyridine]-4-carbimidoyl nitrile 37 as yellow solid.

The invention thus also relates to a process for the manufacture of a compound of formula (I) comprising the sequential steps (a)-(c):

(a) the reaction of a compound of formula (A)

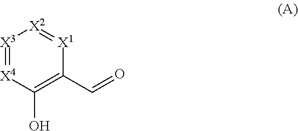

in the presence of a compound of formula (B) and an acid

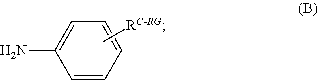

(b) The addition of a cyanide ion source; and

(c) The addition of an oxidizing agent;

wherein X¹ to X⁴ and R^{C} to R^{G} are as defined above.

In step (a), the acid is for example TFA or HCl.

In step (b), the cyanide ion source is for example TMSCN, NaCN or KCN.

In step (c), the oxidizing agent is for example air or MnO₂.

The invention further relates to a compound of formula (I), when manufactured according to a process of the invention.

Pharmaceutical compositions useful herein contain a compound of formula (I), or isomers thereof or pharmaceutically acceptable salts thereof, or metabolites thereof in a pharmaceutically acceptable carrier optionally with other pharmaceutically inert or inactive ingredients.

The pharmaceutical compositions containing a compound of formula (I) may be formulated neat or with one or more pharmaceutical carriers for administration. The amount of the pharmaceutical carrier(s) is determined by the solubility and chemical nature of the compound of formula (I) or an isomer or pharmaceutically acceptable salts, or metabolites thereof, chosen route of administration and standard pharmacological practice. Although the compound of formula (I) or a metabolite thereof, or a pharmaceutically salt thereof or isomer thereof may be administered alone, it may also be administered in the presence of one or more pharmaceutical carriers that are physiologically compatible.

Examples of excipients which may be combined with one or more compound of formula (I) or a metabolite thereof, or isomers thereof or a pharmaceutically acceptable salts thereof include, without limitation, adjuvants, antioxidants, binders, buffers, coatings, coloring agents, compression aids, diluents, disintegrates, emulsifiers, emollients, encapsulating materials, fillers, flavouring agents, glidants, granulating agents, lubricants, metal chelators, osmo-regulators, pH adjustors, preservatives, solubilizes, sorbents, stabilizers, sweeteners, surfactants, suspending agents, syrups, thickening agents, or viscosity regulators. See, for example, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

The compounds of formula (I), or a pharmaceutically acceptable salt, or isomers or a metabolite thereof, and pharmaceutical compositions described herein are useful in treating or regulating diseases or conditions associated with kynurenine pathway. Specifically, the compounds are useful in treating or regulating diseases or conditions associated with increased kynurenine pathway metabolites, for e.g., kynurenine or altered (for example, decreased) tryptophan levels. The compounds are useful for the treatment of disease or condition associated with one or more of indoleamine 2,3-dioxygenase-1 or indoleamine 2,3-disoxygenase-2 or tryptophan 2,3-dioxygenase enzymes.

The utility of the compounds can be illustrated, for example, by their activity in in vitro and in vivo assays known in the art and as described herein. The compounds of formula (I), or a pharmaceutically acceptable salt, or isomers or a metabolite thereof, and pharmaceutical compositions described herein exhibit indoleamine 2,3-dioxygense-1 and/or indoleamine 2,3-disoxygense-2 and/or tryptophan 2,3-dioxygenase inhibitory activity, and decrease the production of kynurenine pathway metabolites. Accordingly, compounds of the invention can be used as therapeutic agents for the treatment of a disease, disorder, or condition directly or indirectly related to or associated with kynurenine pathway metabolites and/or one or more of indoleamine 2,3-dioxygenase-1, indoleamine 2,3-dioxygenase-2 and tryptophan 2,3-dioxygenase enzymes.

Kynurenine pathway associated disease is a disease that can be treated, prevented, ameliorated or cured by reducing kynurenine pathway metabolite levels or increasing tryptophan levels or both. IDO1-, IDO2-, and/or TDO-associated disease can be any disease that can be treated, prevented, ameliorated or cured by regulating enzyme expression and/or activity. The association may be direct or indirect. Accordingly, the compounds described herein are useful for treating diseases associated directly or indirectly with IDO1, IDO2 or TDO or any combination these enzymes, or with kynurenine pathway.

In another aspect, a method for regulating a kynurenine pathway is provided and includes administering compounds of formula (I), or a pharmaceutically acceptable salt, or isomers or a metabolite thereof, and pharmaceutical compositions as described herein to a subject in need thereof. In one aspect, the disease may be any disease treatable by administering a compound of formula (I) or a metabolite thereof, or a pharmaceutically acceptable salt or isomers thereof.

In another aspect, a method of regulating any one or more of any one or more of indoleamine 2,3-dioxygenase-1 or an indoleamine 2,3-dioxygenase-2 or a tryptophan 2,3-dioxygenase enzymes is provided and includes administering a compounds of formula (I), or a pharmaceutically acceptable salt, or isomers or a metabolite thereof, and pharmaceutical compositions as described herein to a subject in need thereof.

In one aspect, a method of reducing kynurenine pathway metabolites is provided and includes compounds of formula (I), or a pharmaceutically acceptable salt, or isomers or a metabolite thereof, and pharmaceutical compositions as described herein to a subject in need thereof.

In another aspect, a method of altering tryptophan levels in a subject and includes administering compounds of formula (I), or a pharmaceutically acceptable salt, or isomers or a metabolite thereof, and pharmaceutical compositions described herein is provided. In one aspect, the tryptophan levels are increased. In another aspect, kynurenine/tryptophan ratio is decreased.

In one aspect, a method of treating a disease associated with or resulting from dysregulation of a kynurenine pathway is provided and includes administering compounds of formula (I), or a pharmaceutically acceptable salt, or isomers or a metabolite thereof, and pharmaceutical compositions thereof as described herein to a subject in need thereof.

In another aspect, a method for treating a disease associated with any one or more of indoleamine 2,3-dioxygenase-1 or indoleamine 2,3-dioxygenase-2 or tryptophan 2,3-dioxygenase enzymes is provided and includes administering compounds of formula (I), or a pharmaceutically acceptable salt, or isomers or a metabolite thereof, and pharmaceutical compositions thereof described herein to a subject in need thereof.

In one aspect, diseases that can be treated using compounds of the invention comprise cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease, or cardiovascular disease. In another aspect, all foregoing methods comprise administration of one or more additional medication or therapeutic agent or therapy. In one aspect, the therapeutic agent is a chemotherapeutic agent selected from a group further comprising a cancer vaccine, a targeted drug, a targeted antibody, an antibody fragment, an antimetabolite, an antineoplastic, an antifolate, a toxin, an alkylating agent, a DNA strand breaking agent, a DNA minor groove binding agent, a pyrimidine analog, a purine analog, a ribonucleotide reductase inhibitor, a tubulin interactive agent, an anti-hormonal agent, an immunomoldulator, an anti-adrenal agent, a cytokine, a radiation therapy, a cell therapy, cell depletion therapy such as B-cell depletion therapy, or a hormone therapy.

In another aspect, a method of treating depression, Alzheimer's disease, dementia, multiple sclerosis, schizophrenia, HIV infection, malaria, rheumatoid arthritis, or insomnia is provided and includes administering compounds of formula (I), or a pharmaceutically acceptable salt, or isomers or a metabolite thereof, and pharmaceutical compositions thereof described herein to a patient.

In yet another aspect, a method for diagnosing and treating a disease associated with kynurenine pathway or any one or more of indoleamine 2,3-dioxygenase-1 or an indoleamine 2,3-dioxygenase-2 or a tryptophan 2,3-dioxygenase enzymes in a subject is provided and includes: (i) assaying a blood and/or tissue sample from a subject; (ii) determining the subject's blood and/or tissue tryptophan or kynurenine concentration or both in the sample; (iii) optionally determining the subject's kynurenine/tryptophan ratio; and (iv) administering compounds of formula (I), or a pharmaceutically acceptable salt, or isomers or a metabolite thereof, and pharmaceutical compositions described herein to a subject.

In still another aspect, a method of monitoring a disease associated with kynurenine pathway or one or more of indoleamine 2,3-dioxygenase-1 or an indoleamine 2,3-dioxygenase-2 or a tryptophan 2,3-dioxygenase enzymes in a subject is provided and includes (i) dosing a subject having a disease associated with kynurenine pathway with compounds of formula (I), or a pharmaceutically acceptable salt, or isomers or a metabolite thereof, and pharmaceutical compositions, (ii) analyzing a blood or tissue sample or both at one or more time points or continuously during a treatment regimen, (iii) determining a tryptophan and a kynurenine concentration in the blood or the tissue sample or both, (iv) optionally determining the subject's kynurenine/tryptophan ratio, and (v) adjusting the treatment regimen or dosage of the compound.

In still a further aspect, a method for treating a disease associated with kynurenine pathway or one or more of an indoleamine 2,3-dioxygenase-1 or an indoleamine 2,3-dioxygenase-2 or a tryptophan 2,3-dioxygenase enzyme in a patient and includes (i) requesting a test providing the results of an analysis to determine whether the patient's kynurenine levels are altered, and (ii) administering compounds of formula (I), or a pharmaceutically acceptable salt, or isomers or a metabolite thereof, and pharmaceutical compositions to the patient if the patient's kynurenine levels are altered.

The compounds of the invention may used in combination with one or more therapeutic agents as described herein. The compounds of the invention are thus useful in the treatment and monitoring the progression of disease associated with kynurenine pathway.

The disease is in particular cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease, or cardiovascular disease

The invention relates in particular to:

A compound or formula (I), in particular of formula (I-F), for use as a therapeutically active substance;

A pharmaceutical composition comprising a compound or formula (I), in particular of formula (I-F), and a therapeutically inert carrier;

The use of a compound or formula (I), in particular of formula (I-F), for the preparation of a medicament for the treatment or prophylaxis of cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease or cardiovascular disease;

A compound or formula (I), in particular of formula (I-F), for use in the treatment or prophylaxis of cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease or cardiovascular disease; and

A method for the treatment or prophylaxis of cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease or cardiovascular disease which method comprises administering an effective amount of a compound or formula (I), in particular of formula (I-F), to a patient in need thereof.

The compound of formula (I), and in particular of formula (I-F), is useful in the treatment or prophylaxis of HBV, for example chronic HBV, HIV, malaria, schizophrenia, depression, HCV, cancer, for example brain tumor of skin cancer, arthritis, for example inflammation-associated arthritis or autoimmune arthritis, allergic airways disease, joint inflammation, multiple sclerosis, Parkinson's disease, Alzheimer's disease, stroke, amyotrophic lateral sclerosis, dementia, allergic encephalomyelitis, Huntington's disease, depression, anxiety, insomnia, atherosclerosis, coronary artery disease or kidney disease, for example chronic kidney disease.

The invention therefore also relates to:

The use of a compound or formula (I), in particular of formula (I-F), for the preparation of a medicament for the treatment or prophylaxis of HBV, for example chronic HBV, HIV, malaria, schizophrenia, depression, HCV, cancer, for example brain tumor of skin cancer, arthritis, for example inflammation-associated arthritis or autoimmune arthritis, allergic airways disease, joint inflammation, multiple sclerosis, Parkinson's disease, Alzheimer's disease, stroke, amyotrophic lateral sclerosis, dementia, allergic encephalomyelitis, Huntington's disease, depression, anxiety, insomnia, atherosclerosis, coronary artery disease or kidney disease, for example chronic kidney disease;

A compound or formula (I), in particular of formula (I-F), for use in the treatment or prophylaxis of HBV, for example chronic HBV, HIV, malaria, schizophrenia, depression, HCV, cancer, for example brain tumor of skin cancer, arthritis, for example inflammation-associated arthritis or autoimmune arthritis, allergic airways disease, joint inflammation, multiple sclerosis, Parkinson's disease, Alzheimer's disease, stroke, amyotrophic lateral sclerosis, dementia, allergic encephalomyelitis, Huntington's disease, depression, anxiety, insomnia, atherosclerosis, coronary artery disease or kidney disease, for example chronic kidney disease; and

A method for the treatment or prophylaxis of HBV, for example chronic HBV, HIV, malaria, schizophrenia, depression, HCV, cancer, for example brain tumor of skin cancer, arthritis, for example inflammation-associated arthritis or autoimmune arthritis, allergic airways disease, joint inflammation, multiple sclerosis, Parkinson's disease, Alzheimer's disease, stroke, amyotrophic lateral sclerosis, dementia, allergic encephalomyelitis, Huntington's disease, depression, anxiety, insomnia, atherosclerosis, coronary artery disease or kidney disease, for example chronic kidney disease.

The following examples are illustrative only and are not intended to limit the present invention. One of skill in the art will recognize that the chemical reactions described may be readily adapted prepare a number of other compounds of the invention, and alternative methods for preparing the compounds of this invention are deemed to be within the scope of this invention. For example, the synthesis of the non-exemplified compounds according to the invention may be successfully performed by modifications apparent to the skilled in the art, e.g., by appropriately protecting the interfering groups, by utilizing other suitable reagents known in the art than those described, and or making routine modifications of reaction conditions. Alternatively, other reactions disclosed herein or known in the art will be recognized as having applicability for preparing other compounds of the invention.

### EXAMPLES

The following abbreviations are used throughout the present examples and specification.

General abbreviations and symbols

| | |
|---|---|
| g | gram |
| mg | milligram |
| ng | nano gram |
| L | liter |
| mL | milliliter |
| mol | mole |
| mmol | millimole |
| min | minutes |
| h | hour |
| ° C. | degrees Celsius |
| EtOAc | Ethyl acetate |
| % | percent |
| μM | micromolar |
| mM | millimolar |
| TLC | thin-layer chromatography |
| HPLC | high-performance liquid chromatography |
| GC-MS | gas chromatography-mass spectrometry |
| LCMS | liquid chromatography-mass spectrometry |
| KYN | Kynurenine |
| SM | starting material |
| eq. | equivalent |
| Pd/C | Palladium on charcoal |
| nM | nanomolar |
| TFE | tetrafluoroethylene |
| RT | room temperature |
| TMSCN | trimethylsilyl cyanide |
| DCM | dichloromethane |
| THF | tetrahydrofurane |
| DMF | dimethylformamide |
| TMEDA | tetramethylethylendiamine |
| TFA | trifluoroacetic acid |
| DMSO | dimethyl sulfoxide |
| LDA | lithiumdiisopropylamine |
| TMSOTf | trimethylsilyl trifluoromethanesulfonate |
| MOM | methoxymehtyl |
| SEM | 2-(trimethylsilyl)ethoxymethyl |
| HMPA | hexamethylphosphoramide |
| TEA | triethanolamine |
| DIPEA | N,N-diisopropylethylamine |
| dba | dibenzylideneacetone |

### Procedure A: Preparation of 2-hydroxyarylimidoylnitrile or Iminonitrile in the Presence of Oxygen

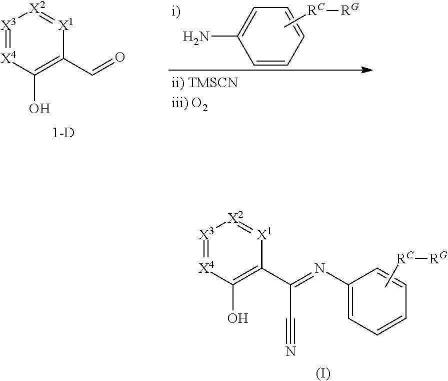

The compound 1-D (1.0 mmol eq.) was dissolved in mixed solvents of TFE and MeCN and then added substituted anilines (1.0 mmol eq.). The resulting mixture was stirred at RT for 1 h. The reaction mass was concentrated and added mixed solvent of DCM and TFE followed by TMSCN (3.5 mmol eq.) at 25° C. The reaction mixture was stirred for 72 h at 25° C. under oxygen balloon. The reaction was monitor by LCMS and after completion of reaction the volatiles were evaporated under reduce pressure to get residue which was purified by column chromatography on silica gel using mixture of suitable solvents of ethyl acetate and hexane to afford iminonitrile (I) as solid.

### Procedure B: Preparation of 2-hydroxyarylimidoylnitrile or Iminonitrile in the Presence of MnO₂

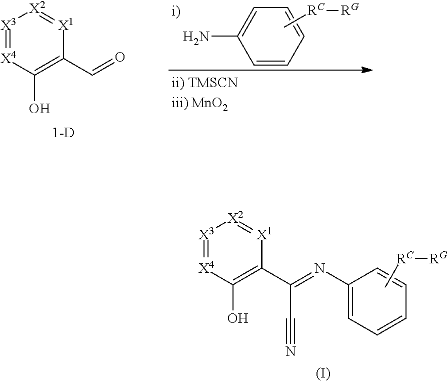

The compound 1-D (1.0 mmol eq.) was dissolved in mixed solvents of TFE and MeCN and then added substituted anilines (1.0 mmol eq.). The resulting mixture was stirred at RT for 1 h. The reaction mass was concentrated and added mixed solvent of DCM and TFE followed by TMSCN (3.5 mmol eq.) at 25° C. The reaction mixture was stirred for 3 h at 25° C., concentrated, and the crude material was dissolved in mixed solvent of chloroform and tetrahydrofuran and then added activated MnO₂ (1.5 mmol eq.) at room temperature and stirred for 3 h. The reaction was monitor by LCMS and after completion of reaction the reaction mass was filtered through celite bed and washed with 10% MeOH in DCM. Filtrate was evaporated under reduce pressure to give crude residue which was purified by column chromatography on silica gel using mixture of suitable solvents of methanol and DCM as eluent. The obtain product was further purified by trituration with 5% ethyl acetate in hexane to afford iminonitrile (I) as solid.

### Example 1

### Synthesis of N-(3-chloro-4-fluorophenyl)-3-hydroxyisonicotinimidoyl nitrile (Compound 01)

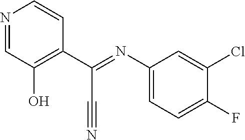 [SMILES: N#C/C(=N\c1ccc(F)c(Cl)c1)c1ccncc1O]

### Step 1: 3-Methoxymethoxy-pyridine

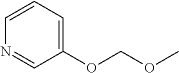

To a stirred solution of 3-hydroxypyridine (60.0 g, 662.9 mmol) in THF:DMF (120:280 mL) at 0° C. was added t-BuOK (81.8 g, 729.28 mmol) portion-wise. After stirring the reaction mixture for 15 min, methoxymethyl chloride (52 mL, 696.13 mmol) was added to it at 0° C. and the resulting mixture was stirred for 1 h at 25° C. After completion of reaction the reaction mixture was diluted with water and extracted with ethyl acetate (4×500 mL). The combined organic layer ware dried over anhydrous sodium sulphate, concentrated under reduced pressure to afford crude material which was purified by column chromatography using silica (100-200 mesh) and 10% EtOAc-hexane as eluent to afford 3-methoxymethoxy-pyridine (54.0 g, 388.48 mmol, 61.5%) as pale brown liquid. LCMS: (M+H) 140

### Step 2: 3-Methoxymethoxy-pyridine-4-carbaldehyde

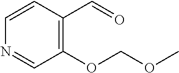

To a stirred solution of 3-(methoxymethoxy)-pyridine (2.0 g, 14.388 mmol) in anhydrous THF (40 mL) was added TMEDA (1.83 g, 15.82 mmol) at 25° C. The reaction mixture was cooled to −78° C., n-BuLi (7.3 mL, 15.82 mmol, 2.17 M in hexane) was added drop-wise manner maintaining the temperature −78° C. After stirring for 2 h at −78° C., DMF (1.52 g, 20.86 mmol) was added to it and stirred for 2 h at 25° C. Reaction mixture was cooled to −40° C. and saturated ammonium chloride solution was added drop wise. The reaction mass was extracted with ethyl acetate (2×250 mL), EtOAc part was washed with water followed by brine, dried over sodium sulphate and concentrated under reduced pressure to afford crude product which was passed through a pad of silica (100-200 mesh) using 10% EtOAc-hexane as eluent to afford 3-methoxymethoxy-pyridine-4-carbaldehyde (1.6 g, 9.57 mmol, 66.6%) as pale yellow liquid. GC-MS: 167 (m/z).

### Step 3: 3-Hydroxy-pyridine-4-carbaldehyde

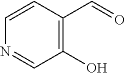

To a stirred solution of 3-methoxymethoxypyridine-4-carbaldehyde (11.0 g, 65.83 mmol) in THF (50 mL) was added 3N HCl (100 mL) and stirred at 60° C. for 1 h. The reaction mixture was cooled under ice bath and pH was adjusted to 7 with solid K₂CO₃. Resulting mixture was extracted with EtOAc (5×250 mL). The organic layer was dried over sodium sulphate, concentrated under reduced pressure to afford crude product which was purified by column chromatography using silica gel (100-200 mesh) and 23% EtOAc/hexane as eluent to afford 3-hydroxy-pyridine-4-carbaldehyde (4.0 g, 32.496 mmol, 49.4%) as pale yellow solid. GC-MS: 123 (m/z), ¹H-NMR (DMSO-d₆, 400 MHz): δ 11.04 (bs, 1H), 10.37 (s, 1H), 8.46 (s, 1H), 8.20 (d, 1H, J=4.88 Hz), 7.46 (d, 1H, J=4.88 Hz). GC-FID: 99.51%.

### Step 4: N-(3-Chloro-4-fluorophenyl)-3-hydroxyisonicotinimidoyl nitrile

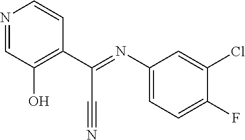 [SMILES: N#C/C(=N\c1ccc(F)c(Cl)c1)c1ccncc1O]

3-Hydroxypyridine-4-carbaldehyde (3.0 g, 24.39 mmol) was taken in mixed solvent (TFE (20 mL):MeCN (20 mL)) and 4-fluoro-3-chloroaniline (3.55 g, 24.39 mmol) was added to it at 25° C. The resulting mixture was stirred at this temperature for 1 h. The reaction mass was concentrated and added mixed solvent [DCM (10 mL):TFE (10 mL)] followed by TMSCN (10.5 mL, 84 mmol) at 25° C. The reaction mixture was stirred for 72 h at 25° C., under oxygen balloon. The reaction was monitor by LCMS and after completion of reaction the volatiles were evaporated under reduce pressure to get residue which was purified by column chromatography on silica gel using 30% ethyl acetate in hexane as eluent to afford N-(3-chloro-4-fluorophenyl)-3-hydroxyisonicotinimidoyl nitrile (1.8 g, 6.545 mmol, 26.7%) as yellow solid.

3-Hydroxypyridine-4-carbaldehyde (3.0 g, 24.39 mmol) was taken in mixed solvent (TFE (20 mL):MeCN (20 mL)) and 4-fluoro-3-chloroaniline (3.55 g, 24.39 mmol) was added to it at 25° C. The resulting mixture was stirred at this temperature for 1 h. The reaction mass was concentrated and added mixed solvent [DCM (10 mL):TFE (10 mL)] followed by TMSCN (10.5 mL, 84 mmol) at 25° C. The reaction mixture was stirred for 3 h at 25° C., concentrated, and the crude material was dissolved in mixed solvent of chloroform (35 mL): tetrahydrofuran (35 mL) and then activated MnO₂ (3.08 g, 35.4 mmol) at room temperature and stirred for 3 h. The reaction was monitor by LCMS and after completion of reaction the reaction mass was filtered through celite bed and washed with 10% MeOH in DCM. Filtrate was evaporated under reduce pressure to give crude residue which was purified by column chromatography on silica gel using 5% methanol in DCM as eluent. The obtain product was further purified by trituration with 5% ethyl acetate in hexane to afford N-(3-chloro-4-fluorophenyl)-3-hydroxyisonicotinimidoyl nitrile (3.8 g, 13.785 mmol, 56.7%) as yellow solid. ¹HNMR: (400 MHz, CD₃CN): δ 11.25 (s, 1H), 8.51 (s, 1H), 8.35 (d, J=5.1 Hz, 1H), 7.71 (d, J=5.1 Hz, 1H), 7.56 (dd, J′=6.5 Hz, J″=2.5 Hz, 1H), 7.44 (t, J=8.8 Hz, 1H), 7.40-7.37 (m, 1H); LCMS: (M+H) 276.

### Example 2

### Synthesis of N-(3,4-difluorophenyl)-3-hydroxy-2′-(methylcarbamoyl)-[2,4′-bipyridine]-4-carbimidoyl nitrile (Compound 37)

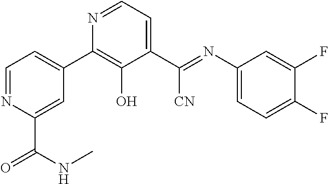 [SMILES: CNC(=O)c1cc(-c2nccc(/C(C#N)=N/c3ccc(F)c(F)c3)c2O)ccn1]

### Step 1: 2-Bromo-3-(methoxymethoxy)pyridine

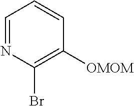

To a stirred solution of 2-bromo-3-hydroxypyridine (50 g, 287.356 mmol) in THF at 0° C. was added t-BuO-K (51.49 g, 459.7 mmol) portion wise. After stirring the reaction mixture for 15 mins, methoxymethyl chloride (34.473 mL, 459.77 mmol) was added to it at 0° C. and the resulting reaction mixture was stirred for 12 h. at 25° C. Reaction mixture was diluted with water and extracted with ethyl acetate (4×500 mL). Organic layer was dried over anhydrous sodium sulfate, concentrated under reduced pressure to afford rude mass which was purified by column chromatography using silica gel (100-200 mesh) and 10% EtOAc-hexane as eluent to afford 2-bromo-3-methoxymethoxy-pyridine (45 g) as pale brown liquid. ¹H-NMR (400 MHz, DMSO-d₆): δ 8.03 (dd, J′=4.5 Hz, J″=1.3 Hz, 1H), 7.60 (dd, J′=8.1 Hz, J″=1.1 Hz, 1H), 7.40 (dd, J′=8.2 Hz, J″=4.5 Hz, 1H), 5.35 (s, 2H), 3.41 (s, 3H).

### Step 2: 2-Bromo-3-(methoxymethoxy)isonicotinaldehyde

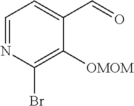

To a stirred solution of 2-Bromo-3-Methoxymethoxypyridine (10.0 g, 45.872 mmol) in anhydrous THF (140 mL) was added LDA (79.5 mL, 59.633 mmol, 0.75 M in THF) at −78° C. After stirring for 1 hr at −78° C., ethylformate (5.559 mL, 68.807 mmol) was added to it and stirred for 30 min at −78° C. The cold bath was removed and the reaction mixture was kept at −10° C. and quenched with aq. NH₄Cl solution (50 mL). Reaction mass was extracted with ethyl acetate (3×150 mL), dried over sodium sulfate and was concentrated under reduced pressure to afford crude mass which was passed through a small pad of silica gel (100-200 mesh) using 4% ethylacetate/hexane as eluent to get 2-bromo-3-methoxymethoxy-pyridine-4-carbaldehyde (5.0 g) as pale yellow solid. ¹H-NMR (400 MHz, DMSO-d₆): δ 10.2 (s, 1H), 8.40 (d, J=4.8 Hz, 1H), 7.67 (d, J=4.8 Hz, 1H), 5.25 (s, 2H), 3.55 (s, 3H).

### Step 3: 4-Formyl-3-(methoxymethoxy)-N-methyl-[2,4′-bipyridine]-2′-carboxamide

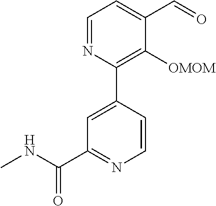

To a stirred solution of 2-bromo-3-methoxymethoxy-pyridine-4-carbaldehyde (5.0 g, 20.325 mmol) in 1,4-dioxane (250 mL) was added crude N-methyl-4-(4,4,5,5-tetramethyl-1,3,2-dioxaborolan-2-yl)picolinamide (3.659 g, 20.325 mmol), K₃PO₄ (27.2 mL, 34.553 mmol, 1.27 M in water) and P(Cy)₃ (1.14 g, 4.065 mmol). The reaction mixture was degassed for 20 min with Argon then added Pd₂(dba)₃ (1.86 g, 2.033 mmol) and again degassed for another 5 min. The reaction mixture was heated to 100° C. for 2 h. After completion of reaction the reaction mixture was cool to room temperature, the volatiles were removed under reduced pressure to afford crude 4-formyl-3-(methoxymethoxy)-N-methyl-[2,4′-bipyridine]-2′-carboxamide (6.3 g), which was forwarded to the next step as such. LCMS: 302 (M+H).

### Step 4: 4-Formyl-3-hydroxy-N-methyl-[2,4′-bipyridine]-2′-carboxamide

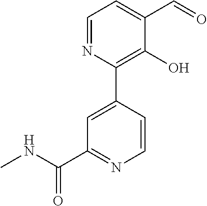 [SMILES: CNC(=O)c1cc(-c2nccc(C=O)c2O)ccn1]

10% TFA-DCM (60 mL) solution was added to crude 4-formyl-3-(methoxymethoxy)-N-methyl-[2,4′-bipyridine]-2′-carboxamide (6.1 g, 20.266 mmol) in DCM (6 mL) at 0° C. After stirring the reaction mixture for 3 h at room temperature, concentrated under reduced pressure, diluted with water and was basified using solid potassium carbonate, washed with ethyl acetate and the aqueous part was acidified to pH-6 using citric acid and extracted with ethyl acetate. The organic layer was washed with brine, dried over anhydrous sodium sulfate and concentrated under reduce pressure to afford crude mass which was purified by trituration using DCM/Et₂O/pentane gave pure 4-formyl-3-hydroxy-N-methyl-[2,4′-bipyridine]-2′-carboxamide (2.8 g) as pale brown solid. ¹H-NMR (400 MHz, DMSO-d₆): δ 11.26 (s, 1H), 10.31 (s, 1H), 8.84 (d, J=4.6 Hz, 1H), 8.75 (d, J=5.0 Hz, 1H), 8.67 (s, 1H), 8.51 (d, J=4.7 Hz, 1H), 8.17 (dd, J′=5.0 Hz, J″=1.6 Hz, 1H), 7.76 (d, J=4.8 Hz, 1H), 2.85 (d, J=4.8 Hz, 3H); LCMS: 258.2 (M+H).

### Step 5: N-(3,4-difluorophenyl)-3-hydroxy-2′-(methylcarbamoyl)-[2,4′-bipyridine]-4-carbimidoyl nitrile

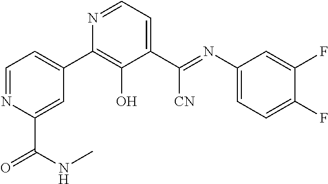 [SMILES: CNC(=O)c1cc(-c2nccc(/C(C#N)=N/c3ccc(F)c(F)c3)c2O)ccn1]

To a stirred solution of 4-formyl-3-hydroxy-N-methyl-[2,4′-bipyridine]-2′-carboxamide (0.2 g, 0.778 mmol) in DCM (3.1 mL) was added 3,4-difluoroaniline (0.077 mL, 0.778 mmol), TMSCN (0.116 g, 1.166 mmol), TMSOTf (0.051 g, 0.233 mmol) at room temperature. The reaction mixture was stirred for 1 hr at 40° C., followed by addition of 10 mmol NH₄OAc buffer (2.3 mL) and further stirred at 40° C. for 20 h. The reaction mixture was filtered through a sintered funnel and washed the solid with MTBE/hexane and dried. The obtained solid material was dissolved in mixed solvent of chloroform (1.0 mL): tetrahydrofuran (1.0 mL) and then activated MnO₂ (0.131 g, 1.517 mmol) at room temperature and stirred for 24 h. The reaction was monitor by TLC and after completion of reaction the reaction mass was filtered through celite bed and washed with 10% MeOH in DCM. Filtrate was evaporated under reduce pressure to give crude residue which was purified by column chromatography on silica gel using 20% EtOAc and hexane as eluent. The obtain product was further purified by trituration with 5% ethyl acetate in hexane to afford N-(3,4-difluorophenyl)-3-hydroxy-2′-(methylcarbamoyl)-[2,4′-bipyridine]-4-carbimido-yl nitrile (0.062 g, 0.157 mmol, 31%) as yellow solid. ¹HNMR: (DMSO-d₆, 500 MHz): δ 12.32 (s, 1H), 8.88 (d, J=4.7 Hz, 1H), 8.78 (d, J=5.05 Hz, 1H), 8.74 (s, 1H), 8.57 (d, J=4.75 Hz, 1H), 8.24 (d, J=5.05 Hz, 1H), 7.87 (d, J=3.8 Hz, 1H), 7.76-7.69 (m, 2H), 7.46-7.44 (m, 1H), 2.86 (d, J=5.05 Hz, 3H); LCMS: (M+H) 394.14.

Compounds of the invention made according to procedures A-B and Examples 1 and 2 as described herein are listed below in TABLE 1. Their characterization is given in TABLE 1A.

TABLE 1

| | | | Proc. |
|---|---|---|---|
| CPD | Chemical Structure | IUPAC Name | Type |
| 01 | 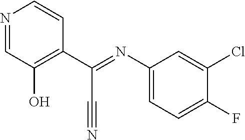 [SMILES: N#C/C(=N\c1ccc(F)c(Cl)c1)c1ccncc1O] | N-(3-Chloro-4- fluorophenyl)-3- hydroxyisonicotini- midoyl nitrile | A, B |
| 02 | 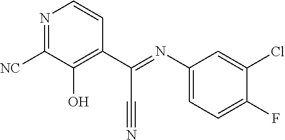 [SMILES: N#C/C(=N\c1ccc(F)c(Cl)c1)c1ccnc(C#N)c1O] | N-(3-Chloro-4- fluorophenyl)-2-cyano- 3- hydroxyIsonicotini- midoyl nitrile | A |
| 03 | 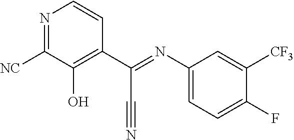 [SMILES: N#C/C(=N\c1ccc(F)c(C(F)(F)F)c1)c1ccnc(C#N)c1O] | 2-Cyano-N-(4-fluoro-3- (trifluoromethyl)phenyl)- 3- hydroxyisonicotini- midoyl nitrile | A |
| 04 | 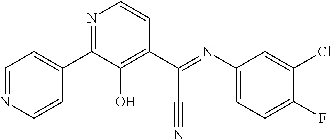 [SMILES: N#C/C(=N\c1ccc(F)c(Cl)c1)c1ccnc(-c2ccncc2)c1O] | N-(3-Chloro-4- fluorophenyl)-3- hydroxy-[2,4′- bipyridine]-4- carbimidoyl nitrile | A |
| 05 | 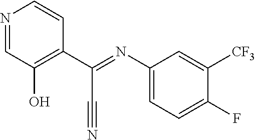 [SMILES: N#C/C(=N\c1ccc(F)c(C(F)(F)F)c1)c1ccncc1O] | N-(4-Fluoro-3- (trifluoromethyl)phenyl)- 3- hydroxyisonicotini- midoyl nitrile | B |
| 06 | 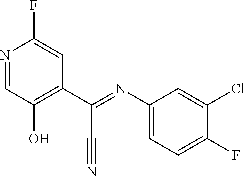 [SMILES: N#C/C(=N\c1ccc(F)c(Cl)c1)c1cc(F)ncc1O] | N-(3-Chloro-4- fluorophenyl)-2-fluoro- 5- hydroxyIsonicotini- midoyl nitrile | B |
| 07 | 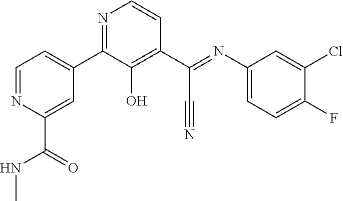 [SMILES: CNC(=O)c1cc(-c2nccc(/C(C#N)=N/c3ccc(F)c(Cl)c3)c2O)ccn1] | N-(3-Chloro-4- fluorophenyl)-3- hydroxy-2′- (methylcarbamoyl)- [2,4′-bipyridine]-4- carbimidoyl nitrile | B |
| 08 | 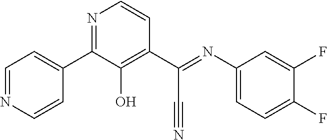 [SMILES: N#C/C(=N\c1ccc(F)c(F)c1)c1ccnc(-c2ccncc2)c1O] | N-(3,4- Difluorophenyl)-3- hydroxy-[2,4′- bipyridine]-4- carbimidoyl nitrile | B |
| 09 | 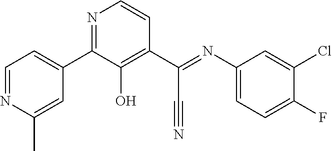 [SMILES: Cc1cc(-c2nccc(/C(C#N)=N/c3ccc(F)c(Cl)c3)c2O)ccn1] | N-(3-Chloro-4- fluorophenyl)-3- hydroxy-2′-methyl- [2,4′-bipyridine]-4- carbimidoyl nitrile | B |
| 10 | 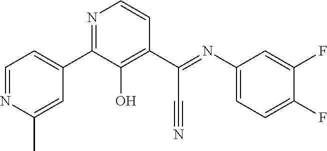 [SMILES: Cc1cc(-c2nccc(/C(C#N)=N/c3ccc(F)c(F)c3)c2O)ccn1] | N-(3,4- Difluorophenyl)-3- hydroxy-2′-methyl- [2,4′-bipyridine]-4- carbimidoyl nitrile | B |
| 11 | 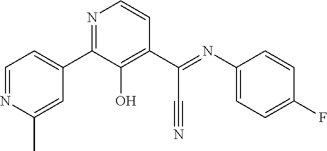 [SMILES: Cc1cc(-c2nccc(/C(C#N)=N/c3ccc(F)cc3)c2O)ccn1] | N-(4-Fluorophenyl)-3- hydroxy-2′-methyl- [2,4′-bipyridine]-4- carbimidoyl nitrile | B |
| 12 | 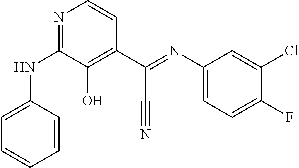 [SMILES: N#C/C(=N\c1ccc(F)c(Cl)c1)c1ccnc(Nc2ccccc2)c1O] | N-(3-Chloro-4- fluorophenyl)-3- hydroxy-2- (phenylamino)isonico- tinimidoyl nitrile | B |
| 13 | 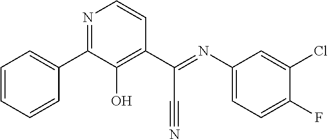 [SMILES: N#C/C(=N\c1ccc(F)c(Cl)c1)c1ccnc(-c2ccccc2)c1O] | N-(3-Chloro-4- fluorophenyl)-3- hydroxy-2- phenylisonicotinimidoyl nitrile | B |
| 14 | 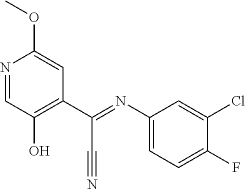 [SMILES: COc1cc(/C(C#N)=N/c2ccc(F)c(Cl)c2)c(O)cn1] | N-(3-Chloro-4- fluorophenyl)-5- hydroxy-2- methoxyisonico- tinimidoyl nitrile | B |
| 15 | 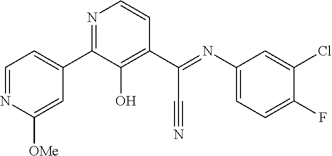 [SMILES: COc1cc(-c2nccc(/C(C#N)=N/c3ccc(F)c(Cl)c3)c2O)ccn1] | N-(3-Chloro-4- fluorophenyl)-3- hydroxy-2′-methoxy- [2,4′-bipyridine]-4- carbimidoyl nitrile | B |
| 16 | 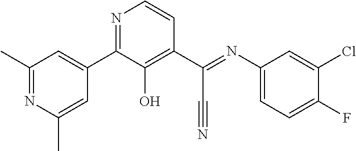 [SMILES: Cc1cc(-c2nccc(/C(C#N)=N/c3ccc(F)c(Cl)c3)c2O)cc(C)n1] | (N-(3-Chloro-4- fluorophenyl)-3- hydroxy-2′,6′-dimethyl- [2,4′-bipyridine]-4- carbimidoyl nitrile | B |
| 17 | 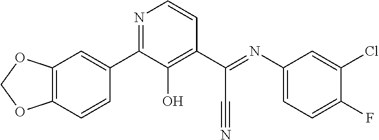 [SMILES: N#C/C(=N\c1ccc(F)c(Cl)c1)c1ccnc(-c2ccc3c(c2)OCO3)c1O] | 2-(Benzo[d][1,3]dioxol- 5-yl)-N-(3-chloro-4- fluorophenyl)-3- hydroxyisonicotini- midoyl nitrile | B |
| 18 | 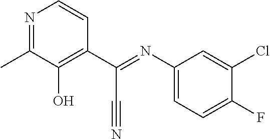 [SMILES: Cc1nccc(/C(C#N)=N/c2ccc(F)c(Cl)c2)c1O] | N-(3-Chloro-4- fluorophenyl)-3- hydroxy-2- methylisonicotinimidoyl nitrile | B |
| 19 | 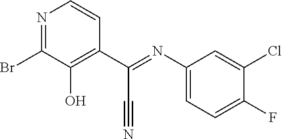 [SMILES: N#C/C(=N\c1ccc(F)c(Cl)c1)c1ccnc(Br)c1O] | 2-Bromo-N-(3-chloro- 4-fluorophenyl)-3- hydroxyIsonicotinimi- doyl nitrile | B |
| 20 | 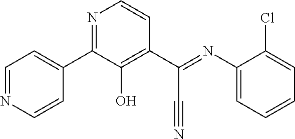 [SMILES: N#C/C(=N\c1ccccc1Cl)c1ccnc(-c2ccncc2)c1O] | (N-(2-Chlorophenyl)-3- hydroxy-[2,4′- bipyridine]-4- carbimidoyl nitrile | B |
| 21 | 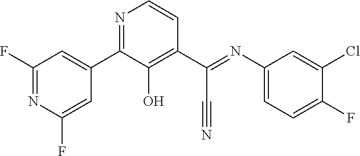 [SMILES: N#C/C(=N\c1ccc(F)c(Cl)c1)c1ccnc(-c2cc(F)nc(F)c2)c1O] | N-(3-Chloro-4- fluorophenyl)-2′,6′- difluoro-3-hydroxy- [2,4′-bipyridine]-4- carbimidoyl nitrile | B |
| 22 | 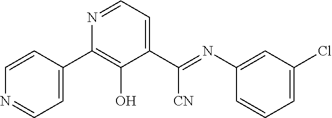 [SMILES: N#C/C(=N\c1cccc(Cl)c1)c1ccnc(-c2ccncc2)c1O] | N-(3-Chlorophenyl)-3- hydroxy-[2,4′- bipyridine]-4- carbimidoyl nitrile | B |
| 23 | 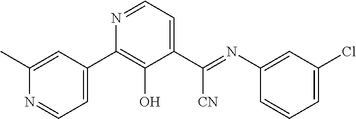 [SMILES: Cc1cc(-c2nccc(/C(C#N)=N/c3cccc(Cl)c3)c2O)ccn1] | N-(3-Chlorophenyl)-3- hydroxy-2′-methyl- [2,4′-bipyridine]-4- carbimidoyl nitrile | B |
| 24 | 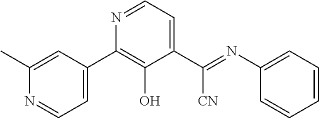 [SMILES: Cc1cc(-c2nccc(/C(C#N)=N/c3ccccc3)c2O)ccn1] | 3-Hydroxy-2′-methyl- N-phenyl-[2,4′- bipyridine]-4- carbimidoyl nitrile | B |
| 25 | 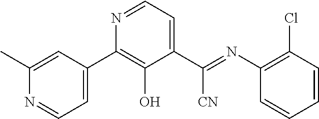 [SMILES: Cc1cc(-c2nccc(/C(C#N)=N/c3ccccc3Cl)c2O)ccn1] | N-(2-Chlorophenyl)-3- hydroxy-2′-methyl- [2,4′-bipyridine]-4- carbimidoyl nitrile | B |
| 26 | 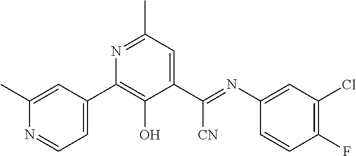 [SMILES: Cc1cc(-c2nc(C)cc(/C(C#N)=N/c3ccc(F)c(Cl)c3)c2O)ccn1] | N-(3-Chloro-4- fluorophenyl)-3- hydroxy-2′,6-dimethyl- [2,4′-bipyridine]-4- carbimidoyl nitrile | B |
| 27 | 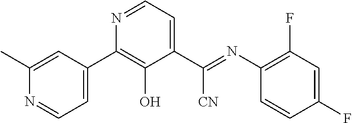 [SMILES: Cc1cc(-c2nccc(/C(C#N)=N/c3ccc(F)cc3F)c2O)ccn1] | N-(2,4- Difluorophenyl)-3- hydroxy-2′-methyl- [2,4′-bipyridine]-4- carbimidoyl nitrile | B |
| 28 | 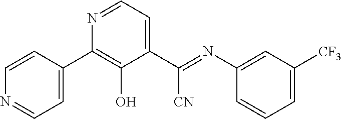 [SMILES: N#C/C(=N\c1cccc(C(F)(F)F)c1)c1ccnc(-c2ccncc2)c1O] | 3-Hydroxy-N-(3- (trifluoromethyl)phenyl)- [2,4′-bipyridine]-4- carbimidoyl nitrile | B |
| 29 | 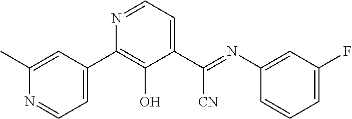 [SMILES: Cc1cc(-c2nccc(/C(C#N)=N/c3cccc(F)c3)c2O)ccn1] | N-(3-Fluorophenyl)-3- hydroxy-2′-methyl- [2,4′-bipyridine]-4- carbimidoyl nitrile | B |
| 30 | 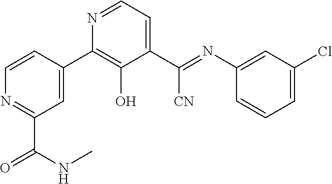 [SMILES: CNC(=O)c1cc(-c2nccc(/C(C#N)=N/c3cccc(Cl)c3)c2O)ccn1] | N-(3-Chlorophenyl)-3- hydroxy-2′- (methylcarbamoyl)- [2,4′-bipyridine]-4- carbimidoyl nitrile | B |
| 31 | 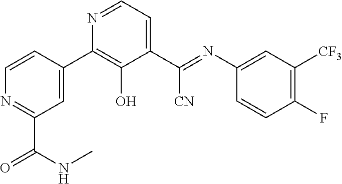 [SMILES: CNC(=O)c1cc(-c2nccc(/C(C#N)=N/c3ccc(F)c(C(F)(F)F)c3)c2O)ccn1] | N-(4-Fluoro-3- (trifluoromethyl)phenyl)- 3-hydroxy-2′- (methylcarbamoyl)- [2,4′-bipyridine]-4- carbimidoyl nitrile | B |
| 32 | 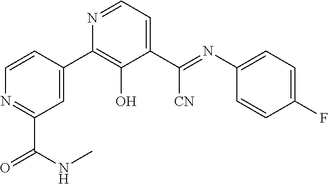 [SMILES: CNC(=O)c1cc(-c2nccc(/C(C#N)=N/c3ccc(F)cc3)c2O)ccn1] | N-(4-Fluorophenyl)-3- hydroxy-2′- (methylcarbamoyl)- [2,4′-bipyridine]-4- carbimidoyl nitrile | B |
| 33 | 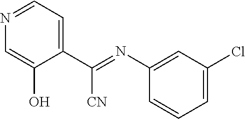 [SMILES: N#C/C(=N\c1cccc(Cl)c1)c1ccncc1O] | N-(3-Chlorophenyl)-3- hydroxyisonicotini- midoyl nitrile | B |
| 34 | 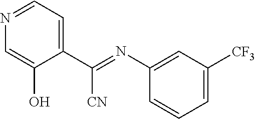 [SMILES: N#C/C(=N\c1cccc(C(F)(F)F)c1)c1ccncc1O] | 3-Hydroxy-N-(3- (trifluoromethyl)phenyl) isonicotinimidoyl nitrile | B |
| 35 | 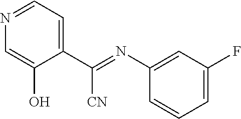 [SMILES: N#C/C(=N\c1cccc(F)c1)c1ccncc1O] | N-(3-Fluorophenyl)-3- hydroxyisonIcotinimi- doyl nitrile | B |
| 36 | 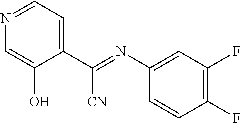 [SMILES: N#C/C(=N\c1ccc(F)c(F)c1)c1ccncc1O] | N-(3,4- Difluorophenyl)-3- hydroxyisonicotinimi- doyl nitrile | B |
| 37 | 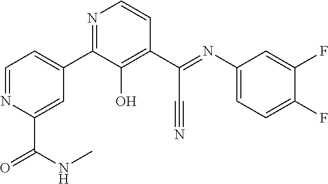 [SMILES: CNC(=O)c1cc(-c2nccc(/C(C#N)=N/c3ccc(F)c(F)c3)c2O)ccn1] | N-(3,4- Difluorophenyl)-3- hydroxy-2′- (methylcarbamoyl)- [2,4′-bipyridine]-4- carbimidoyl nitrile | B |
| 38 | 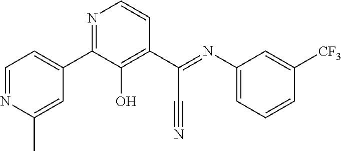 [SMILES: Cc1cc(-c2nccc(/C(C#N)=N/c3cccc(C(F)(F)F)c3)c2O)ccn1] | 3-Hydroxy-2′-methyl- N-(3- (trifluoromethyl)phenyl)- [2,4′-bipyridine]-4- carbimidoyl nitrile | B |
| 39 | 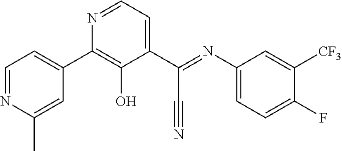 [SMILES: Cc1cc(-c2nccc(/C(C#N)=N/c3ccc(F)c(C(F)(F)F)c3)c2O)ccn1] | N-(4-Fluoro-3- (trifluoromethyl)phenyl)- 3-hydroxy-2′-methyl- [2,4′-bipyridine]-4- carbimidoyl nitrile | B |
| 40 | 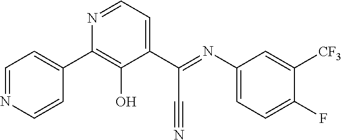 [SMILES: N#C/C(=N\c1ccc(F)c(C(F)(F)F)c1)c1ccnc(-c2ccncc2)c1O] | N-(4-Fluoro-3- (trifluoromethyl)phenyl)- 3-hydroxy-[2,4′- bipyridine]-4- carbimidoyl nitrile | B |
| 41 | 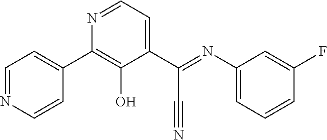 [SMILES: N#C/C(=N\c1cccc(F)c1)c1ccnc(-c2ccncc2)c1O] | N-(3-Fluorophenyl)-3- hydroxy-[2,4′- bipyridine]-4- carbimidoyl nitrile | B |
| 42 | 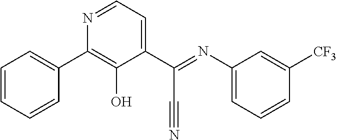 [SMILES: N#C/C(=N\c1cccc(C(F)(F)F)c1)c1ccnc(-c2ccccc2)c1O] | 3-Hydroxy-2-phenyl-N- (3- (trifluoromethyl)phenyl) isonicotinimidoyl nitrile | B |
| 43 | 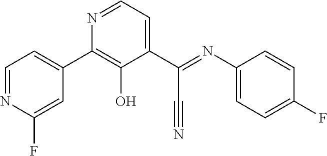 [SMILES: N#C/C(=N\c1ccc(F)cc1)c1ccnc(-c2ccnc(F)c2)c1O] | 2′-Fluoro-N-(4- fluorophenyl)-3- hydroxy-[2,4′- bipyridine]-4- carbimidoyl nitrile | B |
| 44 | 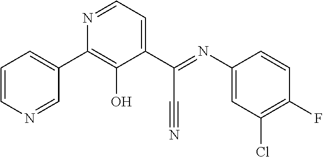 [SMILES: N#C/C(=N\c1ccc(F)c(Cl)c1)c1ccnc(-c2cccnc2)c1O] | N-(3-Chloro-4- fluorophenyl)-3- hydroxy-[2,3′- bipyridine]-4- carbimidoyl nitrile | B |

TABLE 1A

| CPD | ¹H-NMR (400 MHz) proton shift values | LCMS |
|---|---|---|
| 01 | CD₃CN: δ 11.25 (s, 1H), 8.51 (s, 1H), 8.35 (d, J = 5.1 Hz, 1H), 7.71 (d, | [M + H] |
| | J = 5.1 Hz, 1H), 7.56 (dd, J′ = 6.5 Hz, J″ = 2.5 Hz, 1H), 7.44 (t, J = 8.8 Hz, | 276 |
| | 1H), 7.40-7.37 (m, 1H) | |
| 02 | CD₃CN: δ 12.13 (s, 1H), 8.47 (d, J = 5.0 Hz, 1H), 8.00 (d, J = 4.9 Hz, | [M − H] |
| | 1H), 7.64 (d, J = 6.6 Hz, 1H), 7.47-7.46 (m, 2H) | 299.2 |
| 03 | CD₃CN: δ 12.04 (s, 1H), 8.48 (d, J = 5.0 Hz, 1H), 8.02 (d, J = 4.9 Hz, | [M − H] |
| | 1H), 7.82 (d, J = 6.2 Hz, 1H), 7.77-7.75 (m, 1H), 7.55 (t, J = 9.5 Hz, 1H) | 333 |
| 04 | CD₃CN: δ 12.28 (bs, 1H), 8.69 (bs, 2H), 8.51 (d, J = 5.1 Hz, 1H), | [M + H] |
| | 8.01 (d, J = 5.8 Hz, 2H), 7.83 (d, J = 5.0 Hz, 1H), 7.62-7.60 (m, 1H), | 352.9 |
| | 7.48-7.42 (m, 2H) | |
| 05 | CD₃CN: δ 11.23 (s, 1H), 8.53 (s, 1H), 8.36 (d, J = 5.2 Hz, 1H), | [M + H] |
| | 7.75 (dd, J′ = 6.1 Hz, J″ = 2.2 Hz, 1H), 7.72-7.68 (m, 2H), 7.52 (t, = 9.6 Hz, | 310.1 |
| | 1H) | |
| 06 | DMSO-d₆: δ 11.28 (s, 1H), 8.04 (s, 1H), 7.65-7.59 (m, 2H), 7.43 (d, | [M − H] |
| | J = 2.6 Hz, 1H), 7.38-7.34 (m, 1H) | 292 |
| 07 | CD₃CN: δ 12.47 (s, 1H), 8.83 (bs, 1H), 8.72 (bs, 1H), 8.56 (d, J = 5.0 Hz, | [M + H] |
| | 1H), 8.23-8.18 (m, 2H), 7.87 (d, J = 5.0 Hz, 1H), 7.65 (d, J = 6.4 Hz, | 410.2 |
| | 1H), 7.48 (d, J = 7.0 Hz, 2H), 2.98 (d, J = 3.4 Hz, 3H) | |
| 08 | DMSO-d₆: δ 12.26 (s, 1H), 8.71 (d, J = 5.6 Hz, 2H), 8.51 (d, J = 4.8 Hz, | [M + H] |
| | 1H), 8.03 (d, J = 4.9 Hz, 2H), 7.82 (d, J = 4.9 Hz, 1H), 7.73-7.66 (m, | 336.8 |
| | 2H), 7.42-7.40 (m, 1H) | |
| 09 | DMSO-d₆: δ 12.25 (s, 1H), 8.55 (d, J = 5.3 Hz, 1H), 8.45 (d, J = 4.2 Hz, | [M + H] |
| | 1H), 7.90 (s, 1H), 7.83-7.79 (m, 2H), 7.77 (d, J = 5.0 Hz, 1H), 7.64 (t, | 367.1 |
| | J = 9.0 Hz, 1H), 7.53-7.51 (m, 1H), 2.53 (s, 3H) | |
| 10 | DMSO-d₆: δ 12.31 (s, 1H), 8.57 (d, J = 5.3 Hz, 1H), 8.46 (bs, 1H), | [M + H] |
| | 7.93 (s, 1H), 7.86 (s, 1H), 7.80 (d, J = 4.9 Hz, 1H), 7.72-7.66 (m, 2H), | 350.9 |
| | 7.40-7.38 (m, 1H), 2.56 (s, 3H) | |
| 11 | DMSO-d₆: δ 12.56 (s, 1H), 8.57 (d, J = 5.1 Hz, 1H), 8.50 (bs, 1H), | [M + H] |
| | 7.91 (s, 1H), 7.84 (s, 1H), 7.80 (d, J = 4.9 Hz, 1H), 7.63-7.59 (m, 2H), | 333.1 |
| | 7.45 (t, J = 8.7 Hz, 2H), 2.56 (s, 3H) | |
| 12 | CD₃CN: δ 12.08 (s, 1H), 7.89 (d, J = 4.9 Hz, 1H), 7.80 (d, J = 7.9 Hz, | [M + H] |
| | 2H), 7.63 (bs, 1H), 7.57 (dd, J′ = 6.6 Hz, J″ = 2.4 Hz, 1H), 7.44 (t, J = 8.8 Hz, | 367.1 |
| | 1H), 7.41-7.37 (m, 1H), 7.34 (t, J = 7.9 Hz, 2H), 7.14 (d, J = 5.4 Hz, | |
| | 1H), 7.03 (t, J = 7.4 Hz, 1H), | |
| 13 | DMSO-d₆, 500 MHz: δ 12.09 (s, 1H), 8.49 (d, J = 4.1 Hz, 1H), 8.00 (d, | [M + H] |
| | J = 7.55 Hz, 2H), 7.86 (dd, J′ = 6.6 Hz, J″ = 2.2 Hz, 1H), 7.73 (d, J = 5.05 Hz, | 352.0 |
| | 1H), 7.68 (t, J = 9.0 Hz, 1H), 7.59-7.56 (m, 1H), 7.52-7.44 (m, 3H) | |
| 14 | DMSO-d₆, 500 MHz: δ 10.57 (s, 1H), 8.04 (s, 1H), 7.66-7.64 (m, 1H), | [M + H] |
| | 7.61 (d, J = 9.15 Hz, 1H), 7.38-7.35 (m, 1H), 7.07 (s, 1H), 3.84 (s, 3H) | 306.24 |
| 15 | DMSO-d₆, 500 MHz: δ 12.22 (s, 1H), 8.53 (d, J = 5.05 Hz, 1H), | [M + H] |
| | 8.30 (d, J = 5.35 Hz, 1H), 7.86 (dd, J′ = 6.6 Hz, J″ = 2.2 Hz, 1H), 7.83 (d, | 383.0 |
| | J = 5.05 Hz, 1H), 7.69 (t, J = 8.9 Hz, 1H), 7.61-7.57 (m, 2H), 7.44 (s, | |
| | 1H), 3.91 (s, 3H) | |
| 16 | DMSO-d₆, 500 MHz: δ 12.50 (s, 1H), 8.38 (s, 1H), 7.83-7.79 (m, 3H), | [M + H] |
| | 7.77 (d, J = 5.05 Hz, 1H), 7.66 (t, J = 8.97 Hz, 1H), 7.52-7.51 (m, 1H), | 381.1 |
| | 2.51 (s, 6H) | |
| 17 | DMSO-d₆, 500 MHz: δ 12.16 (s, 1H), 8.45 (d, J = 4.75 Hz, 1H), | [M + H] |
| | 7.86 (d, J = 4.65 Hz, 1H), 7.70-7.66 (m, 2H), 7.63 (dd, J′ = 8.2 Hz, J″ = 1.25 Hz, | 396.0 |
| | 1H), 7.58-7.57 (m, 2H), 7.05 (d, J = 8.2 Hz, 1H), 6.10 (s, 2H) | |
| 18 | DMSO-d₆, 500 MHz: δ 11.50 (s, 1H), 8.21 (s, 1H), 7.80 (dd, J′ = 6.6 Hz, | [M + H] |
| | J″ = 2.2 Hz, 1H), 7.66 (t, J = 8.97 Hz, 1H), 7.55 (d, J = 5.05 Hz, 1H), | 390.1 |
| | 7.53-7.50 (m, 1H), 2.50 (s, 3H) | |
| 19 | DMSO-d₆, 500 MHz: δ 11.99 (s, 1H), 8.18 (d, J = 5.05 Hz, 1H), | [M + H] |
| | 7.81 (dd, J′ = 6.6 Hz, J″ = 2.2 Hz, 1H), 7.76 (d, J = 5.0 Hz, 1H), 7.67 (t, J = 8.97 Hz, | 354.06 |
| | 1H), 7.55-7.52 (m, 1H) | |
| 20 | DMSO-d₆, 500 MHz: δ 12.67 (s, 1H), 8.73 (d, J = 5.05 Hz, 2H), | [M + H] |
| | 8.54 (d, J = 4.75 Hz, 1H), 8.06 (d, J = 4.7 Hz, 2H), 7.86 (d, J = 5.0 Hz, 1H), | 335.31 |
| | 7.76 (d, J = 7.9 Hz, 2H), 7.61 (t, J = 7.55 Hz, 1H), 7.51 (t, J = 7.6 Hz, 1H), | |
| 21 | DMSO-d₆, 500 MHz: δ 12.44 (s, 1H), 8.58 (d, J = 5.05 Hz, 1H), | [M + H] |
| | 7.92 (d, J = 5.05 Hz, 1H), 7.88 (dd, J′ = 6.6 Hz, J″ = 2.2 Hz, 1H), 7.80 (s, 2H), | 389.21 |
| | 7.69 (t, J = 8.97 Hz, 1H), 7.61-7.58 (m, 1H) | |
| 22 | DMSO-d₆: δ 12.25 (s, 1H), 8.72 (d, J = 5.2 Hz, 2H), 8.53 (d, J = 4.64 Hz, | [M + H] |
| | 1H), 8.04 (d, J = 4.44 Hz, 2H), 7.83 (d, J = 4.84 Hz, 1H), 7.63-7.60 (m, | 335.0 |
| | 2H), 7.52 (d, J = 7.84 Hz, 1H), 7.45 (d, J = 7.6 Hz, 1H) | |
| 23 | CD₃CN: δ 12.26 s, 1H), 8.57 (d, J = 5.04 Hz, 1H), 8.50 (d, J = 4.96 Hz, | [M + H] |
| | 1H), 7.85 (s, 1H), 7.82-7.79 (m, 2H), 7.57 (t, J = 7.94 Hz, 1H), | 349.1 |
| | 7.50-7.46 (m, 2H), 7.35 (d, J = 7.88 Hz, 1H), 2.58 (s, 3H) | |
| 24 | DMSO-d₆: δ 12.66 (s, 1H), 8.57 (d, J = 5.24 Hz, 1H), 8.51 (bs, 1H), | [M + H] |
| | 7.91 (s, 1H), 7.84 (bs, 1H), 7.81 (d, J = 4.92 Hz, 1H), 7.59 (t, J = 7.66 Hz, | 315.1 |
| | 2H), 7.52-7.45 (m, 3H), 2.56 (s, 3H) | |
| 25 | CD₃CN: δ 12.62 (s, 1H), 8.60 (bs, 1H), 8.50 (d, J = 4.48 Hz, 1H), | [M + H] |
| | 7.89 (s, 1H), 7.83-8.82 (m, 2H), 7.68 (d, J = 7.48 Hz, 1H), 7.61 (d, J = 7.36 Hz, | 349.1 |
| | 1H), 7.55 (t, J = 7.08 Hz, 1H), 7.50-7.48 (m, 1H), 2.60 (s, 3H) | |
| 26 | CD₃CN: δ 12.01 (s, 1H), 8.57 (d, J = 5.0 Hz, 1H), 7.87 (s, 1H), 7.81 (d, | [M + H] |
| | J = 4.64 Hz, 1H), 7.69 (s, 1H), 7.61 (dd, J′ = 6.48 Hz, J″ = 2.16 Hz, 1H), | 381.0 |
| | 7.49-7.41 (m, 2H), 2.63 (s, 3H), 2.59 (s, 3H) | |
| 27 | CD₃CN: δ 12.58 (s, 1H), 8.56 (d, J = 5.0 Hz, 1H), 8.49 (d, J = 4.96 Hz, | [M + H] |
| | 1H), 7.85 (s, 1H), 7.81-7.70 (m, 3H), 7.27-7.17 (m, 2H), 2.73 (s, 3H) | 351.0 |
| 28 | CD₃CN: δ 12.33 (s, 1H), 8.74 (bs, 2H), 8.54 (d, J = 4.84 Hz, 1H), | [M + H] |
| | 8.05 (s, 2H), 7.87 (d, J = 4.84 Hz, 1H), 7.81 (d, J = 4.32 Hz, 2H), 7.76 (s, 1H), | 369.0 |
| | 7.68 (s, 1H) | |
| 29 | DMSO-d₆: δ 12.38 (s, 1H), 8.57 (d, J = 8.04 Hz, 1H), 8.47 (bs, 1H), | [M + H] |
| | 7.93 (s, 1H), 7.86 (s, 1H), 7.81 (d, J = 4.68 Hz, 1H), 7.65-7.60 (m, 1H), | 333.1 |
| | 7.40 (d, J = 9.32 Hz, 1H), 7.33-7.28 (m, 2H), 2.56 (s, 3H) | |
| 30 | DMSO-d₆, 500 MHz: δ 12.34 (s, 1H), 8.88 (q, J = 4.3 Hz, 1H) 8.78 (d, | [M + H] |
| | J = 5.05 Hz, 1H), 8.74 (s, 1H), 8.57 (d, J = 5.05 Hz, 1H), 8.25 (d, J = 5.05 Hz, | 392.21 |
| | 1H), 7.87 (d, J = 5.05 Hz, 1H), 7.65-7.64 (m, 1H), 7.62 (d, J = 8.2 Hz, | |
| | 1H), 7.54 (d, J = 7.85 Hz, 1H), 7.48 (d, J = 7.9 Hz, 1H), 2.86 (d, | |
| | J = 4.75 Hz, 3H) | |
| 31 | DMSO-d₆, 500 MHz: δ 12.23 (s, 1H), 8.88 (d, J = 4.05 Hz, 1H), | [M + H] |
| | 8.77-8.75 (m, 2H), 8.55 (d, J = 4.75 Hz, 1H), 8.25 (s, 1H), 8.05 (d, J = 4.45 Hz, | 444.11 |
| | 1H), 7.93-7.91 (m, 1H), 7.87 (d, J = 4.1 Hz, 1H), 7.79 (t, J = 9.45 Hz, | |
| | 1H), 2.86 (d, J = 4.75 Hz, 3H) | |
| 32 | DMSO-d₆, 500 MHz: δ 12.65 (s, 1H), 8.87 (s, 1H), 8.78-8.75 (m, 2H), | [M + H] |
| | 8.57-8.56 (m, 1H), 8.25 (s, 1H), 7.86 (t, J = 5.05 Hz, 1H), 7.66-7.63 (m, | 376.28 |
| | 2H), 7.47 (t, J = 8.02 Hz, 2H), 2.86 (d, J = 4.75 Hz, 3H) | |
| 33 | DMSO-d₆, 500 MHz: δ 11.33 (s, 1H), 8.47 (s, 1H), 8.28 (d, J = 5.05 Hz, | [M + H] |
| | 1H), 7.68 (d, J = 5.05 Hz, 1H), 7.57 (t, J = 8.35 Hz, 1H), | 258.01 |
| | 7.45-7.44 (m, 2H), 7.27 (d, J = 8.2 Hz, 1H) | |
| 34 | CD₃CN, 500 MHz: δ 11.19 (s, 1H), 8.56 (s, 1H), 8.39 (d, J = 5.4 Hz, | [M + H] |
| | 1H), 7.79 (d, J = 5.05 Hz, 2H), 7.76 (d, J = 5.35 Hz, 1H), 7.72 (s, 1H), | 292.13 |
| | 7.66-7.64 (m, 1H) | |
| 35 | CD₃CN, 500 MHz: δ 11.00 (s, 1H), 8.55 (s, 1H), 8.38 (d, J = 5.05 Hz, | [M + H] |
| | 1H), 7.75 (d, J = 5.05 Hz, 1H), 7.63-7.59 (m, 1H), 7.26-7.22 (m, 2H), | 242.13 |
| | 7.20-7.18 (m, 1H) | |
| 36 | CD₃CN, 500 MHz: δ 10.75 (s, 1H), 8.54 (s, 1H), 8.38 (d, J = 5.05 Hz, | [M + H] |
| | 1H), 7.74 (d, J = 5.05 Hz, 1H), 7.49 (q, J = 9.25 Hz, 1H), 7.43-7.39 (m, | 260.16 |
| | 1H), 7.28-7.27 (m, 1H) | |
| 37 | DMSO-d₆, 500 MHz: δ 12.32 (s, 1H), 8.88 (d, J = 4.7 Hz, 1H), 8.78 (d, | [M + H] |
| | J = 5.05 Hz, 1H), 8.74 (s, 1H), 8.57 (d, J = 4.75 Hz, 1H), 8.24 (d, J = 5.05 Hz, | 394.14 |
| | 1H), 7.87 (d, J = 3.8 Hz, 1H), 7.76-7.69 (m, 2H), 7.46-7.44 (m, 1H), | |
| | 2.86 (d, J = 5.05 Hz, 3H) | |
| 38 | DMSO-d₆, 500 MHz: δ 12.33 (s, 1H), 8.58 (d, J = 5.05 Hz, 1H), | [M + H] |
| | 8.48 (d, J = 4.75 Hz, 1H), 7.96 (s, 1H), 7.91 (s, 1H), 7.89 (d, J = 3.8 Hz, 1H), | 383.31 |
| | 7.82 (bs, 3H), 7.78-7.76 (m, 1H), 2.57 (s, 3H) | |
| 39 | CD₃CN: δ 12.25 (s, 1H), 8.57 (d, J = 5.0 Hz, 1H), 8.50 (d, J = 4.92 Hz, | [M + H] |
| | 1H), 7.85-7.73 (m, 5H), 7.54 (t, J = 9.48 Hz, 1H), 2.58 (s, 3H) | 401.0 |
| 40 | CD₃CN: δ 12.26 (s, 1H), 8.70 (d, J = 5.16, 2H), 8.52 (d, J = 4.96 Hz, | [M + H] |
| | 1H), 8.01 (d, J = 5.56 Hz, 2H), 7.83 (d, J = 4.96 Hz, 1H), 7.80-7.79 (m, | 387.0 |
| | 1H), 7.75-7.73 (m, 1H), 7.54 (t, J = 9.50 Hz, 1H) | |
| 41 | DMSO-d₆: δ 12.30 (s, 1H), 8.72 (d, J = 5.72 Hz, 2H), 8.52 (s, 1H), | [M + H] |
| | 8.04 (s, 2H), 7.83 (d, J = 4.92 Hz, 1H), 7.66-7.61 (m, 1H), 7.41 (d, J = 9.36 Hz, | 319.0 |
| | 1H), 7.34-7.29 (m, 2H). | |
| 42 | CD₃CN: δ 12.15 (s, 1H), 8.47 (d, J = 5.12 Hz, 1H), 8.01 (d, J = 6.88 Hz, | [M + H] |
| | 2H), 7.78-7.72 (m, 4H), 7.65 (s, 1H), 7.52-7.43 (m, 3H) | 368.0 |
| 43 | CD₃CN: δ 12.76 (s, 1H), 8.51 (d, J = 4.84 Hz, 1H), 8.32 (d, J = 5.04 Hz, | [M + H] |
| | 1H), 8.03 (d, J = 4.52 Hz, 1H), 7.86 (d, J = 4.8 Hz, 1H), 7.76 (s, 1H), | 337.0 |
| | 7.54-7.51 (m, 2H), 7.34 (t, J = 8.6 Hz, 2H) | |
| 44 | CD₃CN: δ 12.24 (s, 1H), 9.21 (s, 1H), 8.62-8.61 (m, 1H), 8.49 (d, | [M + H] |
| | J = 5.0 Hz, 1H), 8.38-8.36 (m, 1H), 7.79 (d, J = 5.0 Hz, 1H), 7.60 (dd, | 353.0 |
| | J′ = 6.34 Hz, J″ = 1.82 Hz, 1H), 7.48-7.44 (m, 3H) | |

TABLE 2 is a non-exhaustive list of compounds of the invention that are made using the procedures described herein.

TABLE 2

| CPD | Structure | IUPAC Name | M + 1 |
|---|---|---|---|
| 45 | 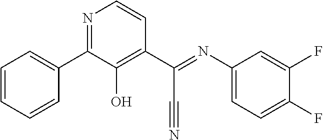 [SMILES: N#C/C(=N\c1ccc(F)c(F)c1)c1ccnc(-c2ccccc2)c1O] | N-(3,4-Difluorophenyl)-3- hydroxy-2- phenylisonicotinimidoyl nitrile | 336.09 |
| 46 | 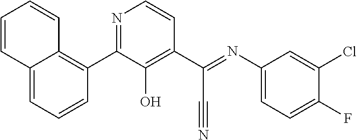 [SMILES: N#C/C(=N\c1ccc(F)c(Cl)c1)c1ccnc(-c2cccc3ccccc23)c1O] | N-(3-Chloro-4-fluorophenyl)- 3-hydroxy-2-(naphthalen-1- yl)isonicotinimidoyl nitrile | 402.08 |
| 47 | 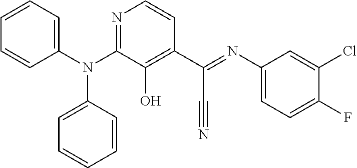 [SMILES: N#C/C(=N\c1ccc(F)c(Cl)c1)c1ccnc(N(c2ccccc2)c2ccccc2)c1O] | N-(3-Chloro-4-fluorophenyl)- 2-(diphenylamino)-3- hydroxyisonicotinimidoyl nitrile | 443.1 |
| 48 | 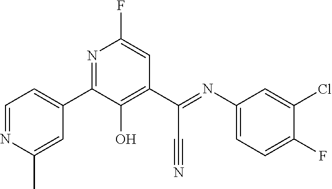 [SMILES: Cc1cc(-c2nc(F)cc(/C(C#N)=N/c3ccc(F)c(Cl)c3)c2O)ccn1] | N-(3-Chloro-4-fluorophenyl)- 6-fluoro-3-hydroxy-2′-methyl- [2,4′-bipyridine]-4-carbimidoyl nitrile | 385.06 |
| 49 | 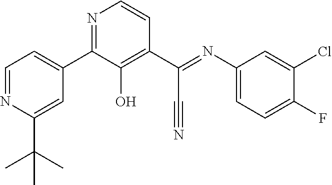 [SMILES: CC(C)(C)c1cc(-c2nccc(/C(C#N)=N/c3ccc(F)c(Cl)c3)c2O)ccn1] | 2′-(tert-Butyl)-N-(3-chloro-4- fluorophenyl)-3-hydroxy-[2,4′- bipyridine]-4-carbimidoyl nitrile | 409.12 |
| 50 | 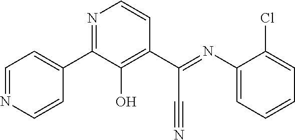 [SMILES: N#C/C(=N\c1ccccc1Cl)c1ccnc(-c2ccncc2)c1O] | N-(2-Chlorophenyl)-3- hydroxy-[2,4′-bipyridine]-4- carbimidoyl nitrile | 335.07 |
| 51 | 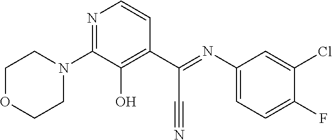 [SMILES: N#C/C(=N\c1ccc(F)c(Cl)c1)c1ccnc(N2CCOCC2)c1O] | N-(3-Chloro-4-fluorophenyl)- 3-hydroxy-2- morpholinoisonicotinimidoyl nitrile | 361.08 |
| 52 | 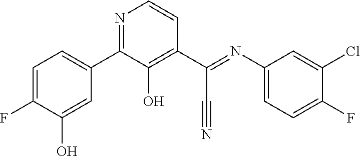 [SMILES: N#C/C(=N\c1ccc(F)c(Cl)c1)c1ccnc(-c2ccc(F)c(O)c2)c1O] | N-(3-Chloro-4-fluorophenyl)- 2-(4-fluoro-3-hydroxyphenyl)- 3-hydroxyisonicotinimidoyl nitrile | 386.05 |
| 53 | 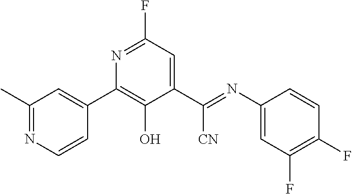 [SMILES: Cc1cc(-c2nc(F)cc(/C(C#N)=N/c3ccc(F)c(F)c3)c2O)ccn1] | N-(3,4-Difluorophenyl)-6- fluoro-3-hydroxy-2′-methyl- [2,4′-bipyridine]-4-carbimidoyl nitrile | 369.09 |
| 54 | 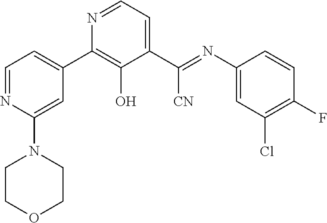 [SMILES: N#C/C(=N\c1ccc(F)c(Cl)c1)c1ccnc(-c2ccnc(N3CCOCC3)c2)c1O] | N-(3-Chloro-4-fluorophenyl)- 3-hydroxy-2′-morpholino-[2,4′- bipyridine]-4-carbimidoyl nitrile | 438.11 |
| 55 | 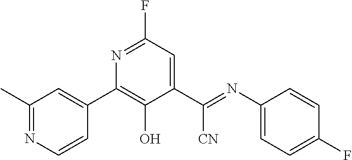 [SMILES: Cc1cc(-c2nc(F)cc(/C(C#N)=N/c3ccc(F)cc3)c2O)ccn1] | 6-Fluoro-N-(4-fluorophenyl)- 3-hydroxy-2′-methyl-[2,4′- bipyridine]-4-carbimidoyl nitrile | 351.10 |
| 56 | 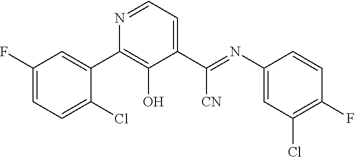 [SMILES: N#C/C(=N\c1ccc(F)c(Cl)c1)c1ccnc(-c2cc(F)ccc2Cl)c1O] | N-(3-Chloro-4-fluorophenyl)- 2-(2-chloro-5-fluorophenyl)-3- hydroxyisonicotinimidoyl nitrile | 404.01 |
| 57 | 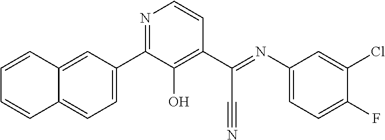 [SMILES: N#C/C(=N\c1ccc(F)c(Cl)c1)c1ccnc(-c2ccc3ccccc3c2)c1O] | N-(3-Chloro-4-fluorophenyl)- 3-hydroxy-2-(naphthalen-2- yl)isonicotinimidoyl nitrile | 402.08 |
| 58 | 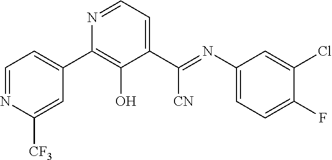 [SMILES: N#C/C(=N\c1ccc(F)c(Cl)c1)c1ccnc(-c2ccnc(C(F)(F)F)c2)c1O] | N-(3-Chloro-4-fluorophenyl)- 3-hydroxy-2′-(trifluoromethyl)- [2,4′-bipyridine]-4-carbimidoyl nitrile | 421.04 |
| 59 | 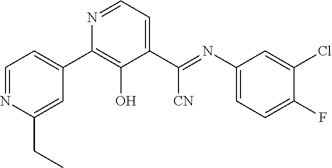 [SMILES: CCc1cc(-c2nccc(/C(C#N)=N/c3ccc(F)c(Cl)c3)c2O)ccn1] | N-(3-Chloro-4-fluorophenyl)- 2′-ethyl-3-hydroxy-[2,4′- bipyridine]-4-carbimidoyl nitrile | 381.09 |
| 60 | 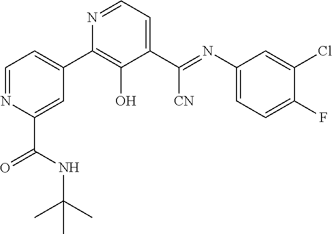 [SMILES: CC(C)(C)NC(=O)c1cc(-c2nccc(/C(C#N)=N/c3ccc(F)c(Cl)c3)c2O)ccn1] | 2′-(tert-Butylcarbamoyl)-N-(3- chloro-4-fluorophenyl)-3- hydroxy-[2,4′-bipyridine]-4- carbimidoyl nitrile | 452.12 |
| 61 | 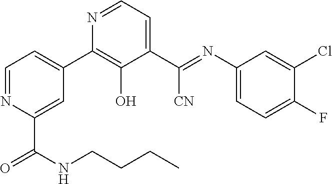 [SMILES: CCCCNC(=O)c1cc(-c2nccc(/C(C#N)=N/c3ccc(F)c(Cl)c3)c2O)ccn1] | 2′-(Butylcarbamoyl)-N-(3- chloro-4-fluorophenyl)-3- hydroxy-[2,4′-bipyridine]-4- carbimidoyl nitrile | 452.12 |
| 62 | 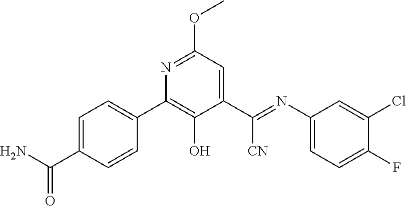 [SMILES: COc1cc(/C(C#N)=N/c2ccc(F)c(Cl)c2)c(O)c(-c2ccc(C(N)=O)cc2)n1] | 2-(4-Carbamoylphenyl)-N-(3- chloro-4-fluorophenyl)-3- hydroxy-6- methoxyisonicotinimidoyl nitrile | 425.08 |
| 63 | 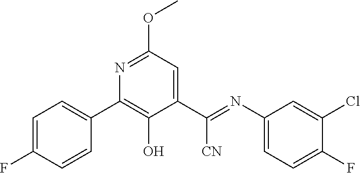 [SMILES: COc1cc(/C(C#N)=N/c2ccc(F)c(Cl)c2)c(O)c(-c2ccc(F)cc2)n1] | N-(3-Chloro-4-fluorophenyl)- 2-(4-fluorophenyl)-3-hydroxy- 6-methoxyisonicotinimidoyl nitrile | 400.06 |
| 64 | 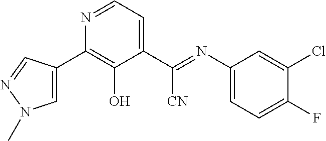 [SMILES: Cn1cc(-c2nccc(/C(C#N)=N/c3ccc(F)c(Cl)c3)c2O)cn1] | N-(3-Chloro-4-fluorophenyl)- 3-hydroxy-2-(1-methyl-1H- pyrazol-4-yl)isonicotinimidoyl c nitrile | 356.07 |
| 65 | 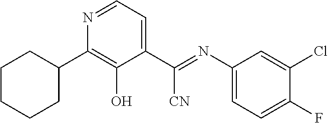 [SMILES: N#C/C(=N\c1ccc(F)c(Cl)c1)c1ccnc(C2CCCCC2)c1O] | N-(3-Chloro-4-fluorophenyl)- 2-cyclohexyl-3- hydroxyisonicotinimidoyl nitrile | 358.11 |
| 66 | 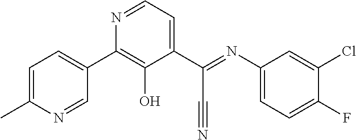 [SMILES: Cc1ccc(-c2nccc(/C(C#N)=N/c3ccc(F)c(Cl)c3)c2O)cn1] | N-(3-Chloro-4-fluorophenyl)- 3-hydroxy-6′-methyl-[2,3′- bipyridine]-4-carbimidoyl nitrile | 367.07 |
| 67 | 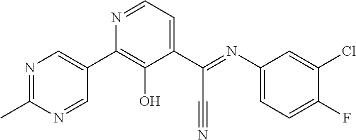 [SMILES: Cc1ncc(-c2nccc(/C(C#N)=N/c3ccc(F)c(Cl)c3)c2O)cn1] | N-(3-Chloro-4-fluorophenyl)- 3-hydroxy-2-(2- methylpyrimidin-5- yl)isonicotinimidoyl nitrile | 368.07 |
| 68 | 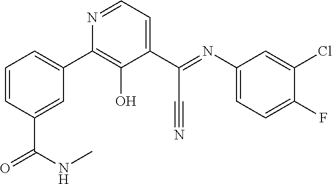 [SMILES: CNC(=O)c1cccc(-c2nccc(/C(C#N)=N/c3ccc(F)c(Cl)c3)c2O)c1] | N-(3-Chloro-4-fluorophenyl)- 3-hydroxy-2-(3- (methylcarbamoyl)phenyl) isonicotinimidoyl nitrile | 409.08 |
| 69 | 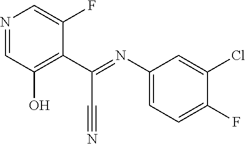 [SMILES: N#C/C(=N\c1ccc(F)c(Cl)c1)c1c(O)cncc1F] | N-(3-chloro-4-fluorophenyl)-3- fluoro-5- hydroxyisonicotinimidoyl nitrile | 294.02 |

### Example 3

### Reduction of LPS Induced Plasma Kynurenine Levels in C57BL/6 Mice

Inflammatory mediators such as Lipopolysaccharides (LPS) and Interferon-gamma (IFNg) are well-established inducers of IDO1 expression. Intraperitoneal (i.p.) administration of bacterial lipopolysaccharide (LPS) induces peak IDO1 activity in a variety of tissues within one day after LPS administration resulting in the production and release of kynurenine into the bloodstream (Takikawa, O., et al. (1986) J. Biol. Chem. 261:3648-53; Yoshida, H., et al. (1998) Cell 94:739-750). LPS-injected mice have been used as models to study IDO1 expression and activity. Three—eight fed C57 BL/6 mice (age 7-8 weeks, weight: about 20-22 g) were injected intrapritoneally with bacterial lipopolysaccharide (LPS; 26:B6 Sigma) at a concentration of 6 mg/kg. Animals were then housed in normal condition for 20 hours at which time the test compounds were administered orally in formulation containing 30% polyethylene glycol 400 (PEG 400) and 20% propylene glycol (PG) in normal saline (Dosing volume 10 mL/kg). Blood was drawn through retro-orbital bleeds into a tube containing 100 mM EDTA for plasma collection at the following times: just prior to LPS treatment, just prior to test compound dosing (0 hr) and then at 2 hr, 4 hr, 6 hr, 8 hr, 24 hr and 48 hr post-test compound dosing. Plasma KYN and drug levels were determined by LC/MS/MS using an API4000 mass spectrometer (Applied Biosystems) coupled to a Shimadzu Prominence LC system fitted with a C18 column.

Representative compounds of the invention were tested as described above and the data is shown in TABLE 3. In vivo pharmacodynamics studies with LPS-injected mouse model show that the compounds of the invention inhibit the activity of IDO1 and reduce plasma kynurenine metabolite, KYN levels in vivo. The percentage of decrease of kynurenine level at two hours is given in TABLE 3. The compounds of the invention decrease kynurenine levels. The compounds of the invention trigger a decrease of kynurenine levels at 2 hrs of at least 5%.

TABLE 3

| Compound | % |
|---|---|
| 01 | 73 |
| 02 | 45 |
| 03 | 25 |
| 04 | 77 |
| 05 | 64 |
| 06 | 73 |
| 07 | 43 |
| 08 | 83 |
| 09 | 71 |
| 10 | 79 |
| 11 | 82 |
| 12 | 15 |
| 13 | 46 |
| 14 | 18 |
| 15 | 47 |
| 16 | 36 |
| 17 | 10 |
| 18 | 53 |
| 19 | 38 |
| 20 | 46 |
| 21 | 21 |
| 22 | 87 |
| 23 | 79 |
| 24 | 81 |
| 25 | 37 |
| 26 | 11 |
| 27 | 64 |
| 28 | 70 |
| 29 | 75 |
| 30 | 64 |
| 31 | 43 |
| 32 | 67 |
| 33 | 80 |
| 34 | 67 |
| 35 | 74 |
| 36 | 79 |
| 37 | 68 |
| 38 | 69 |
| 39 | 67 |
| 40 | 77 |
| 41 | 81 |
| 42 | 32 |
| 43 | 69 |
| 44 | 77 |

### Example A

### Film Coated Tablets Containing the Following Ingredients can be Manufactured in a Conventional Manner

| Ingredients | Per tablet | |
|---|---|---|
| Kernel: | | |
| Compound of formula (I) | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titan dioxide | 0.8 mg | 1.6 mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

### Capsules Containing the Following Ingredients can be Manufactured in a Conventional Manner

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

### Injection Solutions can have the Following Composition

| Compound of formula (I) | 3.0 | mg |
|---|---|---|
| Polyethylene glycol 400 | 150.0 | mg |
| Acetic acid | q.s. ad pH 5.0 | |
| Water for injection solutions | ad 1.0 | ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 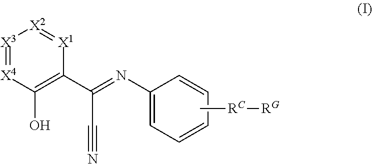
wherein:
X¹ is CR¹, N, or NO;
X² is CR², N, or NO;
X³ is CR³, N, or NO;
X⁴ is CR⁴, N, or NO;
wherein at least one of X¹, X², X³ and X⁴ is N;
R¹, R², R³ and R⁴ are independently selected from the group consisting of H, optionally substituted C₁-C₆ alkyl, optionally substituted C₂-C₆ alkenyl, optionally substituted C₂-C₆ alkynyl, optionally substituted C₁-C₆ alkoxy, mono or bicyclic optionally substituted C₆-C₁₄ aryl, mono or bicyclic optionally substituted heteroaryl, optionally substituted (aryl)alkyl, (alkoxy)carbonyl, (alkyl)amido, (alkyl)amino, optionally substituted mono or bicyclic cycloalkyl, optionally substituted mono or bicyclic heterocyclyl, aminoalkyl, alkylcarboxyl, (alkyl)carboxyamido, optionally substituted (aryl)amino, hydroxyl, halogen, C₁-C₆ haloalkyl, optionally substituted heterocyclyl(alkyl)-, optionally substituted heteroaryl(alkyl), hydroxyalkyl, perfluoroalkyl, optionally substituted aryloxy, optionally substituted heteroaryloxy, optionally substituted C₃-C₈ cycloalkoxy, N(R⁵)₂, CN, NO₂, CO₂H, CONR^{A}R^{B}, S(O)ₙR⁵, and optionally substituted heterocyclyloxy having 1 to 2 heteroatoms selected from the group consisting of O, S(O)ₙ, and NR⁶;
n is 0 to 2;
R^{A} and R^{B} are independently selected from the group consisting of H, optionally substituted C₁-C₆ alkyl, optionally substituted mono or bicyclic C₆-C₁₄ aryl, optionally substituted mono or bicyclic heteroaryl, optionally substituted (aryl)alkyl, optionally substituted mono or bicyclic C₃-C₈ cycloalkyl, optionally substituted mono or bicyclic heterocyclyl, C₁-C₆ haloalkyl, optionally substituted heterocyclyl(alkyl), optionally substituted heteroaryl(alkyl), hydroxyalkyl, and perfluoroalkyl;
R⁵ is independently selected from the group consisting H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or heterocyclyloxy having 1 to 2 heteroatoms selected from the group consisting of O, S(O)ₙ, and NR⁶;
R⁶ is independently selected from the group consisting of H, C₁-C₆ alkyl, mono or bicyclic C₆-C₁₄ aryl, mono or bicyclic heteroaryl, (aryl)alkyl, (alkoxy)carbonyl, (alkyl)amido, (alkyl)amino, mono or bicyclic cycloalkyl, mono or bicyclic heterocyclyl, alkylcarboxyl, heterocyclyl(alkyl), heteroaryl(alkyl), hydroxyalkyl, perfluoroalkyl, aryloxy, heteroaryloxy, C₃-C₆ cycloalkoxy, or optionally substituted heterocyclyloxy; and
R^{C} to R^{G} are independently selected from the group consisting of H, halogen, C₁-C₆ haloalkyl, C₁-C₆ alkoxy, heterocycle, optionally substituted C₁-C₆ alkyl, C₃-C₃ cycloalkyl, CN, —O(aryl), C₂-C₆ alkynyl, C(O)C₁-C₆ alkyl, —O—C₁-C₆ haloalkyl, and optionally substituted aryl;
or an isomer thereof, or a metabolite thereof, or a pharmaceutically acceptable salt or ester thereof.

2. The compound according to claim 1 of formula (I-F) 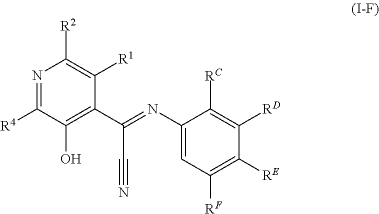
wherein
R¹ is hydrogen or halogen;
R² is hydrogen, halogen, alkyl or alkoxy;
R⁴ is hydrogen, halogen, alkyl, cycloalkyl, cyano, pyridinyl, alkylpyridinyl, alkylaminocarbonylpyridinyl, alkoxypyridinyl, alkylpyridinyl, halopyridinyl, morpholinylpyridinyl, haloalkylpyridinyl, phenyl, halohydroxyphenyl, halophenyl, phenylamino, diphenylamino, aminocarbonylphenyl, naphthyl, benzo[d][1,3]dioxolyl, morpholinyl, alkylpyrazolyl or alkylpyrimidinyl;
R^{C} is hydrogen or halogen;
R^{D} is hydrogen, halogen or haloalkyl;
R^{E} is hydrogen or halogen; and
R^{F} is hydrogen or halogen;
or a pharmaceutically acceptable salt or ester thereof.

3. The compound according to claim 1, wherein R¹ is hydrogen or fluoro.

4. The compound according to claim 1, wherein R¹ is hydrogen.

5. The compound according to claim 1, wherein R² is hydrogen, fluoro, methyl or methoxy.

6. The compound according to claim 1, wherein R² is hydrogen.

7. The compound according to claim 1, wherein R⁴ is alkylpyridinyl or alkylaminocarbonylpyridinyl.

8. The compound according to claim 1, wherein R⁴ is methylpyridinyl or methylaminocarbonylpyridinyl.

9. The compound according to claim 1, wherein R^{C} is hydrogen, chloro or fluoro.

10. The compound according to claim 1, wherein R^{C} is hydrogen.

11. The compound according to claim 1, wherein R^{D} is hydrogen or halogen.

12. The compound according to claim 1, wherein R^{D} is hydrogen, chloro or fluoro.

13. The compound according to claim 1, wherein R^{E} is halogen.

14. A The compound according to claim 1, wherein R^{E} is fluoro.

15. The compound according to claim 1, wherein R^{F} is hydrogen, chloro or fluoro.

16. The compound according to claim 1, wherein R^{F} is hydrogen.

17. The compound according to claim 1, selected from the group consisting of:
N-(3-Chloro-4-fluorophenyl)-3-hydroxyisonicotinimidoyl nitrile;
N-(3-Chloro-4-fluorophenyl)-2-cyano-3-hydroxyisonicotinimidoyl nitrile;
2-Cyano-N-(4-fluoro-3-(trifluoromethyl)phenyl)-3-hydroxyisonicotinimidoyl nitrile;
N-(3-Chloro-4-fluorophenyl)-3-hydroxy-[2,4′-bipyridine]-4-carbimidoyl nitrile;
N-(4-Fluoro-3-(trifluoromethyl)phenyl)-3-hydroxyisonicotinimidoyl nitrile;
N-(3-Chloro-4-fluorophenyl)-2-fluoro-5-hydroxyIsonicotinimidoyl nitrile;
N-(3-Chloro-4-fluorophenyl)-3-hydroxy-2′-(methylcarbamoyl)-[2,4′-bipyridine]-4-carbimidoyl nitrite;
N-(3,4-Difluorophenyl)-3-hydroxy-[2,4′-bipyridine]-4-carbimidoyl nitrite;
N-(3-Chloro-4-fluorophenyl)-3-hydroxy-2′-methyl-[2,4-bipyridine]-4-carbimidoyl nitrile;
N-(3,4-Difluorophenyl)-3-hydroxy-2′-methyl-[2,4′-bipyridine]-4-carbimidoyl nitrile;
N-(4-Fluorophenyl)-3-hydroxy-2′-methyl-[2,4′-bipyridine]-4-carbimidoyl nitrile;
N-(3-Chloro-4-fluorophenyl)-3-hydroxy-2-(phenylamino)isonicotinimidoyl nitrile;
N-(3-Chloro-4-fluorophenyl)-3-hydroxy-2-phenylisonicotinimidoyl nitrile,
N-(3-Chloro-4-fluorophenyl)-S-hydroxy-2-methoxyisonicotinimidoyl nitrite;
N-(3-Chloro-4-fluorophenyl)-3-hydroxy-2′-methoxy-[2,4′-bipyridine]-4-carbimidoyl nitrile;
(N-(3-Chloro-4-fluorophenyl)-3-hydroxy-2′,6′-dimethyl-[2,4′-bipyridine]-4-carbimidoyl nitrile;
2-(Benzo[d][1,3]dioxol-5-yl)-N-(3-chloro-4-fluorophenyl)-3-hydroxyisonicotinimidoyl nitrile;
N-(3-Chloro-4-fluorophenyl)-3-hydroxy-2-methylisonicotinimidoyl nitrile;
2-Bromo-N-(3-chloro-4-fluorophenyl)-3-hydroxyIsonicotinimidoyl nitrile;
(N-(2-Chlorophenyl)-3-hydroxy-[2,4′-bipyridine]-4-carbimidoyl nitrile;
N-(3-Chloro-4-fluorophenyl)-2′,6′-difluoro-3-hydroxy-[2,4′-bipyridine]-4-carbimidoyl nitrile;
N-(3-Chlorophenyl)-3-hydroxy-[2,4′-bipyridine]-4-carbimidoyl nitrile;
N-(3-Chlorophenyl)-3-hydroxy-2′-methyl-[2,4′-bipyridine]-4-carbimidoyl nitrile;
3-Hydroxy-2′-methyl-N-phenyl-[2,4′-bipyridine]-4-carbimidoyl nitrile;
N-(2-Chlorophenyl)-3-hydroxy-2′-methyl-[2,4′-bipyridine]-4-carbimidoyl nitrile;
N-(3-Chloro-4-fluorophenyl)-3-hydroxy-2′,6-dimethyl-[2,4-bipyridine]-4-carbimidoyl nitrile;
N-(2,4-Difluorophenyl)-3-hydroxy-2′-methyl-[2,4′-bipyridine]-4-carbimidoyl nitrile;
3-Hydroxy-N-(3-(trifluoromethyl)phenyl)-[2,4′-bipyridine]-4-carbimidoyl nitrile;
N-(3-Fluorophenyl)-3-hydroxy-2′-methyl-[2,4′-bipyridine]-4-carbimidoyl nitrile;
N-(3-Chlorophenyl)-3-hydroxy-2′-(methylcarbamoyl)-[2,4′-bipyridine]-4-carbimidoyl nitrile;
N-(4-Fluoro-3-(trifluoromethyl)phenyl)-3-hydroxy-2′-(methylcarbamoyl)-[2,4′-bipyridine]-4-carbimidoyl nitrile;
N-(4-Fluorophenyl)-3-hydroxy-2′-(methylcarbamoyl)-[2,4′-bipyridine]-4-carbimidoyl nitrile;
N-(3-Chlorophenyl)-3-hydroxyisonicotinimidoyl nitrile;
3-Hydroxy-N-(3-(trifluoromethyl)phenyl)isonicotinimidoyl nitrile;
N-(3-Fluorophenyl)-3-hydroxyisonIcotinimiidoyl nitrile;
N-(3,4-Difluorophenyl)-3-hydroxyisonicotinimidoyl nitrile;
N-(3,4-Difluorophenyl)-3-hydroxy-2′-(methylcarbamoyl)-[2,4′-bipyridine]-4-carbimidoyl nitrite;
3-Hydroxy-2′-methyl-N-(3-(trifluoromethyl)phenyl)-[2,4′-bipyridine]-4-carbimidoyl nitrile;
N-(4-Fluoro-3-(trifluoromethyl)phenyl)-3-hydroxy-2′-methyl-[2,4′-bipyridine]-carbimidoyl nitrile;
N-(4-Fluoro-3-(trifluoromethyl)phenyl)-3-hydroxy-[2,4′-bipyridine]-4-carbimidoyl nitrile;
N-(3-Fluorophenyl)-3-hydroxy-[2,4′-bipyridine]-4-carbimidoyl nitrile;
3-Hydroxy-2-phenyl-N-(3-(trifluoromethyl)phenyl)isonicotinimidoyl nitrile;
2′-Fluoro-N-(4-fluorophenyl)-3-hydroxy-[2,4′-bipyridine]-4-carbimidoyl nitrile;
N-(3-Chloro-4-fluorophenyl)-3-hydroxy-[2,3′-bipyridine]-4-carbimidoyl nitrile;
N-3,4-Difluorophenyl)-3-hydroxy-2-phenylisonicotinimidoyl nitrile;
N-(3-Chloro-4-fluorophenyl)-3-hydroxy-2-(naphthalen-1-yl)isonicotinimidoyl nitrile;
N-(3-Chloro-4-fluorophenyl)-2-(diphenylamino)-3-hydroxyisonicotinimidoyl nitrile;
N-(3-Chloro-4-fluorophenyl)-6-fluoro-3-hydroxy-2′-methyl-[2,4′-bipyridine]-4-carbimidoyl nitrile;
2′-(tert-Butyl)-N-(3-chloro-4-fluorophenyl)-3-hydroxy-[2,4′-bipyridine]-4-carbimidoyl nitrile;
N-(2-Chlorophenyl)-3-hydroxy-[2,4′-bipyridine]-4-carbimidoyl nitrile;
N-(3-Chloro-4-fluorophenyl)-3-hydroxy-2-morpholinoisonicotinimidoyl nitrile;
N-(3-Chloro-4-fluorophenyl)-2-(4-fluoro-3-hydroxyphenyl)-3-hydroxyisonicotinimidoyl nitrile;
N-(3,4-Difluorophenyl)-6-fluoro-3-hydroxy-2′-methyl-[2,4′-bipyridine]-4-carbimidoyl nitrile;
N-(3-Chloro-4-fluorophenyl)-3-hydroxy-2′-morpholino-[2,4′-bipyridine]-4-carbimidoyl nitrile;
6-Fluoro-N-(4-fluorophenyl)-3-hydroxy-2′-methyl-[2,4′-bipyridine]-4-carbimidoyl nitrile;
N-(3-Chloro-4-fluorophenyl)-2-(2-chloro-5-fluorophenyl)-3-hydroxyisonicotinimidoyl nitrile;
N-(3-Chloro-4-fluorophenyl)-3-hydroxy-2-(naphthalen-2-yl)isonicotinimidoyl nitrile;
N-(3-Chloro-4-fluorophenyl)-3-hydroxy-2′-(trifluoromethyl)-[2,4′-bipyridine]-4-carbimidoyl nitrile;
N-(3-Chloro-4-fluorophenyl)-2′-ethyl-3-hydroxy-[2,4′-bipyridine]-4-carbimidoyl nitrile;
2′-(tert-Butylcarbamoyl)-N-(3-chloro-4-fluorophenyl)-3-hydroxy-[2,4′-bipyridine]-4-carbimidoyl nitrile;
2′-(Butylcarbamoyl)-N-(3-chloro-4-fluorophenyl)-3-hydroxy-[2,4′-bipyridine]-4-carbimidoyl nitrile;
2-(4-Carbamoylphenyl)-N-(3-chloro-4-fluorophenyl)-3-hydroxy-6-methoxyisonicotinimidoyl nitrile;
N-(3-Chloro-4-fluorophenyl)-2-(4-fluorophenyl)-3-hydroxy-6-methoxyisonicotinimidoyl nitrile;
N-(3-Chloro-4-fluorophenyl)-3-hydroxy-2-(1-methyl-1H-pyrazol-4-yl)isonicotinimidoyl c nitrile;
N-(3-Chloro-4-fluorophenyl)-2-cyclohexyl-3-hydroxyisonicotinimidoyl nitrile;
N-(3-Chloro-4-fluorophenyl)-3-hydroxy-6′-methyl-[2,3′-bipyridine]-4-carbimidoyl nitrile;
N-(3-Chloro-4-fluorophenyl)-3-hydroxy-2-(2-methylpyrimidin-5-yl)isonicotinimidoyl nitrile;
N-(3-Chloro-4-fluorophenyl)-3-hydroxy-2-(3-(methylcarbamoyl)phenyl)isonicotinimidoyl nitrile; and
N-(3-chloro-4-fluorophenyl)-3-fluoro-5-hydroxyisonicotinimidoyl nitrile.

18. A process for the manufacture of a compound according to claim 1, comprising the sequential steps (a)-(c)
(a) reacting a compound of formula (A) 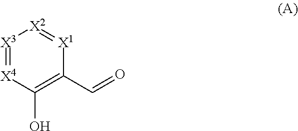
in the presence of a compound of formula (B) and an acid 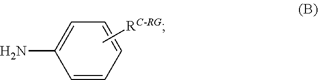
(b) adding an nitrile ion source; and
(c) adding an oxidizing agent;
wherein X¹ to X⁴ and R^{C} to R^{G} are as defined in claim 1.

19-20. (canceled)

21. A pharmaceutical composition, comprising a therapeutically effective amount of a compound according to claim 1, or an isomer thereof, or a metabolite thereof, or pharmaceutically acceptable salt or ester thereof, and a therapeutically inert carrier.

22-23. (canceled)

24. A method for the treatment or prophylaxis of cancer, bacterial infection, viral infection, parasitic infection, immune-mediated disorder, autoimmune disorder, inflammatory disease, central nervous system disease, peripheral nervous system disease, neurodegenerative disease, mood disorder, sleep disorder, cerebrovascular disease, peripheral artery disease or cardiovascular disease, comprising the step of administering a therapeutically effective amount of a compound according to claim 1, or an isomer thereof, or a metabolite thereof, or a pharmaceutically acceptable salt or ester thereof, to a patient in need thereof.

25. (canceled)
